(19) United States
(12) Patent Application Publication
(10) Pub. No.: US 2024/0427175 A1
(43) Pub. Date: Dec. 26, 2024

(54) LENS, METHOD, AND APPARATUS FOR ALTERATION OF COLOR PERCEPTION AND DISCRIMINABILITY

(71) Applicant: Donald Keith Roper, Logan, UT (US)

(72) Inventor: Donald Keith Roper, Logan, UT (US)

(73) Assignee: Utah State University, Logan, UT (US)

(21) Appl. No.: 18/752,584

(22) Filed: Jun. 24, 2024

Related U.S. Application Data
(60) Provisional application No. 63/522,997, filed on Jun. 23, 2023.

(51) Int. Cl. G02C 7/10; A61B 3/06; B29D 11/00; G02C 7/02; G02C 7/04

(52) U.S. Cl. CPC G02C 7/104; A61B 3/066; B29D 11/00038; G02C 7/027; G02C 7/04

(57) ABSTRACT

Examples of the present disclosure include a lens. The lens includes a body of a contact lens. The body has a side. The lens includes a plurality of nodes formed onto the side. Each of the plurality of nodes has a parameter value that is based on a reduction of transmission of light through the lens at one or more wavelengths selected to alter a color perception and/or a color discriminability of a wearer of the lens. The parameter value includes a value of: a distance between the node and an adjacent node of the plurality of nodes, a dimension of the node, a configuration of an arrangement in which the plurality of nodes are arranged on the side, a composition of a material coating the node, and/or a material composition of the node.

### CROSS-REFERENCES TO RELATED APPLICATIONS

This application claims the benefit of U.S. Provisional Patent Application No. 63/522,997 entitled “LENS, METHOD, AND APPARATUS FOR IMPROVEMENT OF COLOR PERCEPTION AND DISCERNMENT BASED ON COLOR VISION DEFICITS” and filed on Jun. 23, 2023 for Keith Roper, which is incorporated herein by reference.

### GOVERNMENT SUPPORT

This invention was made with government support under grant no. 1R15EY035066-01 awarded by the National Institute of Health. The government has certain rights in this invention.

### FIELD

This invention relates to alteration of color perception and/or discrimination between colors and more particularly relates to a lens, method, and apparatus for altering color perception and/or discriminability at particular wavelengths of light.

### BACKGROUND

Perception of colors and discrimination between colors helps to convey information visually and affects aesthetic experience and performance in occupational, academic, and personal settings.

### SUMMARY

Examples of the present disclosure include a lens. The lens includes a body of a contact lens. The body includes a side. The lens includes a plurality of nodes formed onto the side. Each node of the plurality of nodes has a parameter value that is based at least in part on a reduction of transmission of light through the lens at one or more wavelengths, the one or more wavelengths selected to alter a color perception and/or a color discriminability of a person. The parameter value is a value of: a distance between the node and an adjacent node of the plurality of nodes, a shape of the node, a dimension of the node, a configuration of an arrangement in which the plurality of nodes are arranged on the side, a composition of a material coating the node, and/or a material composition of the node.

Examples of the present disclosure include an apparatus. The apparatus includes a perception module configured to determine a person's perception of light at one or more wavelengths across a range of tested wavelengths. The apparatus includes a discriminability module configured to determine a person's discriminability between light across the range of tested wavelengths. The apparatus includes a transmission reduction module configured to determine a wavelength range selected to alter a color perception and/or a color discriminability of the person based at least in part on the person's perception of light, the person's discriminability between light across the range of tested wavelengths, and the one or more wavelengths. The apparatus includes a node module configured to determine, for each node of a plurality of nodes and based at least in part on the wavelength range, a parameter value. The parameter value includes: a distance between the node and an adjacent node of the plurality of nodes, a shape of the node, a dimension of the node, a configuration of an arrangement of nodes of the plurality of nodes, a composition of a material coating a node, and/or a material composition of the node. The apparatus includes a formation module configured to perform the following: form the node based at least in part on the shape of the node, the dimension of the node, the composition of the material coating the node, and/or the material composition of the node; form a plurality of cavities in a substrate based at least in part on the distance between the node and the adjacent node of the plurality of nodes and/or the configuration of the arrangement of nodes of the plurality of nodes; and/or cause the plurality of nodes to be deposited onto a substrate based at least in part on the distance between the node and the adjacent node of the plurality of nodes and/or the configuration of the arrangement of nodes of the plurality of nodes. At least a portion of said modules include one or more of hardware circuits, programmable hardware circuits and executable code, the executable code stored on one or more computer readable storage media.

Examples of the present disclosure include a method. The method includes determining, using a perception module, a person's perception of light at one or more wavelengths across a range of tested wavelengths. The method includes determining, using a discriminability module, a person's discriminability between light across the range of tested wavelengths. The method includes determining, using a transmission reduction module, a wavelength range to alter the color perception and/or color discriminability of the person based at least in part on the perception of light, the discriminability between light, and the one or more wavelengths. The method includes determining, using a node module, for each node of a plurality of nodes and based at least in part on the wavelength range, a parameter value, the parameter value comprising: a distance between the node and an adjacent node of the plurality of nodes, a shape of the node, a dimension of the node, a configuration of an arrangement of nodes of the plurality of nodes, a composition of a material coating a node, and/or a material composition of the node. The method includes performing at least one of the following: forming the node based at least in part on the shape of the node, the dimension of the node, the composition of the material coating the node, and/or the material composition of the node; forming a plurality of cavities in a substrate based at least in part on the distance between the node and the adjacent node of the plurality of nodes and/or the configuration of the arrangement of nodes of the plurality of nodes; and/or causing the plurality of nodes to be deposited onto a substrate based at least in part on the distance between the node and the adjacent node of the plurality of nodes and/or the configuration of the arrangement of nodes of the plurality of nodes.

### BRIEF DESCRIPTION OF THE DRAWINGS

In order that the advantages of the invention will be readily understood, a more particular description of the invention briefly described above will be rendered by reference to specific examples that are illustrated in the appended drawings. Understanding that these drawings depict only typical examples of the invention and are not therefore to be considered to be limiting of its scope, the invention will be described and explained with additional specificity and detail through the use of the accompanying drawings, in which:

FIG. 1 is a front view of one example of a lens, according to the present disclosure;

FIG. 2 is a front view of one example of a lens having a hexagonal array of nodes, according to the present disclosure;

FIG. 3A is a side view of one example of a lens, according to the present disclosure;

FIG. 3B is a cross-sectional view of one example of a lens, according to the present disclosure;

FIG. 4 illustrates a graph of transmission of light as a function of wavelength of light for one example of a lens according to the present disclosure;

FIG. 5A is a schematic block diagram illustrating one example of an apparatus for alteration of color discriminability, according to the present disclosure;

FIG. 5B is a schematic block diagram illustrating another example of an apparatus for alteration of color discriminability, according to the present disclosure;

FIG. 6 is a schematic block diagram illustrating an example of a system for alteration of color discriminability, according to the present disclosure;

FIG. 7 illustrates a graph of transmission as a function of wavelength for simulations and/or lenses of the present disclosure of different materials, according to one or more examples of the present disclosure;

FIG. 8 illustrates a graph of transmission as a function of wavelength for simulations and/or lenses of the present disclosure of different node shapes, according to one or more examples of the present disclosure;

FIG. 9 illustrates a graph of transmission as a function of wavelength for simulations and/or lenses of the present disclosure of different aspect ratios, according to one or more examples of the present disclosure;

FIG. 10 illustrates a graph of transmission as a function of wavelength for simulations and/or lenses of the present disclosure of different pitch spacing between nodes, according to one or more examples of the present disclosure;

FIG. 11 is a flow chart diagram of an example of a method of making a lens, according to the present disclosure;

FIGS. 12A-H illustrate an example of a method of forming a stamp for forming a lens, according to the present disclosure;

FIG. 13 illustrates an example of depositing nodes into a lens, according to the present disclosure;

FIG. 14 is a graph of cone sensitivities as a function of wavelength, according to one or more examples of the present disclosure;

FIGS. 15A-D illustrate an example of a method of forming a lens, according to the present disclosure;

FIG. 16 is a flow chart of an example of a method of coating a mold, according to the present disclosure;

FIG. 17 is a flow chart of an example of another method of coating a mold, according to the present disclosure; and

FIGS. 18A-E are schematic diagrams illustrating a method of coating a mold, according to the present disclosure.

### DETAILED DESCRIPTION

Reference throughout this specification to “one example,” “an example,” or similar language means that a particular feature, structure, or characteristic described in connection with the example is included in at least one example. Thus, appearances of the phrases “in one example,” “in an example,” and similar language throughout this specification may, but do not necessarily, all refer to the same example, but mean “one or more but not all examples” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more examples. In the following description, numerous specific details are provided, such as examples of programming, software modules, user selections, network transactions, database queries, database structures, hardware modules, hardware circuits, hardware chips, etc., to provide a thorough understanding of examples of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one example of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Reference throughout this specification to “one example,” “an example,” or similar language means that a particular feature, structure, or characteristic described in connection with the example is included in at least one example. Thus, appearances of the phrases “in one example,” “in an example,” and similar language throughout this specification may, but do not necessarily, all refer to the same example, but mean “one or more but not all examples” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the examples may be combined in any suitable manner. One skilled in the relevant art will recognize that the examples may be practiced without one or more of the specific features or advantages of a particular example. In other instances, additional features and advantages may be recognized in certain examples that may not be present in all examples.

These features and advantages of the examples will become more fully apparent from the following description and appended claims, or may be learned by the practice of examples as set forth hereinafter. As will be appreciated by one skilled in the art, aspects of the present invention may be embodied as a system, method, and/or computer program product. Accordingly, aspects of the present invention may take the form of an entirely hardware example, an entirely software example (including firmware, resident software, micro-code, etc.) or an example combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom very large scale integrated (“VLSI”) circuits or gate arrays, off-the-shelf semiconductors such as logic chips, transistors, or other discrete components. A module may also be implemented in programmable hardware devices such as a field programmable gate array (“FPGA”), programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (“ISA”) instructions, machine instructions, machine dependent instructions, microcode, firmware instructions, state-setting data, or either source code or object code written in any combination of one or more programming languages, including an object oriented programming language such as Python, Java, Smalltalk, C++ or the like, and conventional procedural programming languages, such as the “C” programming language, Fortran, or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (“LAN”) or a wide area network (“WAN”), or the connection may be made to an external computer (for example, through the Internet using an Internet Service Provider). In some examples, electronic circuitry including, for example, programmable logic circuitry, field-programmable gate arrays (“FPGA”), or programmable logic arrays (“PLA”) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to examples of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program instructions may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

The schematic flowchart diagrams and/or schematic block diagrams in the Figures illustrate the architecture, functionality, and operation of possible implementations of apparatuses, systems, methods and computer program products according to various examples of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding examples. Indeed, some arrows or other connectors may be used to indicate only the logical flow of the depicted example. For instance, an arrow may indicate a waiting or monitoring period of unspecified duration between enumerated steps of the depicted example. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of” includes one and only one of any single item in the list.

Examples of the present disclosure include a lens. The lens includes a body of a contact lens. The body includes a side. The lens includes a plurality of nodes formed onto the side. Each node of the plurality of nodes has a parameter value that is based at least in part on a reduction of transmission of light through the lens at one or more wavelengths, the one or more wavelengths selected to alter a color perception and/or a color discriminability of a person. The parameter value is a value of: a distance between the node and an adjacent node of the plurality of nodes, a shape of the node, a dimension of the node, a configuration of an arrangement in which the plurality of nodes are arranged on the side, a composition of a material coating the node, and/or a material composition of the node.

In some examples, the one or more wavelengths include a wavelength range that is based at least in part on a color vision deficiency (“CVD”) profile of the person. In some examples, the plurality of nodes are separated from each other at a distance that is based at least in part on a wavelength within the wavelength range. In some examples, the CVD profile includes a deficiency of the person in perceiving light within a tested wavelength range of light and a deficiency of the person in discriminating between light of different wavelengths within the tested wavelength range of light. In some examples, the wavelength range is based at least in part on the deficiency of the person in perceiving light within the tested wavelength range of light and the deficiency of the person in discriminating between light of different wavelengths within the tested wavelength range of light.

In some examples, each of the plurality of nodes include an ellipse shape. The ellipse shape includes an aspect ratio. The dimension includes the aspect ratio.

In some examples, the plurality of nodes are separated from each other a distance not less than 10 nanometers (“nm”) and not greater than 1000 nm away from each other.

In some examples, the body includes a plurality of cavities and each node of the plurality of nodes is located within a cavity of the plurality of cavities.

In some examples, at least one node of the plurality of nodes includes an ellipse shape. In some examples, a length of a minor axis of the ellipse shape of the at least one node is not less than 5 nm and not greater than 150 nm.

In some examples, the arrangement of the plurality of nodes is a substantially square array on the side. The side includes a convex side.

In some examples, each node of the plurality of nodes includes a metallic material.

Examples of the present disclosure include an apparatus. The apparatus includes a perception module configured to determine a person's perception of light at one or more wavelengths across a range of tested wavelengths. The apparatus includes a discriminability module configured to determine a person's discriminability between light across the range of tested wavelengths. The apparatus includes a transmission reduction module configured to determine a wavelength range selected to alter a color perception and/or a color discriminability of the person based at least in part on the person's perception of light, the person's discriminability between light across the range of tested wavelengths, and the one or more wavelengths. The apparatus includes a node module configured to determine, for each node of a plurality of nodes and based at least in part on the wavelength range, a parameter value. The parameter value includes: a distance between the node and an adjacent node of the plurality of nodes, a shape of the node, a dimension of the node, a configuration of an arrangement of nodes of the plurality of nodes, a composition of a material coating a node, and/or a material composition of the node. The apparatus includes a formation module configured to perform the following: form the node based at least in part on the shape of the node, the dimension of the node, the composition of the material coating the node, and/or the material composition of the node; form a plurality of cavities in a substrate based at least in part on the distance between the node and the adjacent node of the plurality of nodes and/or the configuration of the arrangement of nodes of the plurality of nodes; and/or cause the plurality of nodes to be deposited onto a substrate based at least in part on the distance between the node and the adjacent node of the plurality of nodes and/or the configuration of the arrangement of nodes of the plurality of nodes. At least a portion of said modules include one or more of hardware circuits, programmable hardware circuits and executable code, the executable code stored on one or more computer readable storage media.

In some examples, the transmission reduction module is further configured to determine a percentage of transmission reduction of light within the wavelength range. The node module is further configured to determine the value of the parameter based at least in part on the percentage.

In some examples, the apparatus includes a simulation module configured to generate a number of simulations of transmission of light through a lens, each simulation of the number of simulations simulating nodes of a test value of one or more test values of the parameter. The node module is further configured to determine the value of the parameter based at least in part on results from the number of simulations.

In some examples, the node module is further configured to determine the value of the parameter based at least in part on the person's perception of light across the range of tested wavelengths and on the person's discriminability between light of different wavelengths across the range of tested wavelengths.

In some examples, the substrate includes a contact lens body, a substrate of a stamp for forming cavities in a contact lens body, a substrate of a stamp for forming a mold for forming a contact lens, and/or a mold for forming a contact lens.

Examples of the present disclosure include a method. The method includes determining, using a perception module, a person's perception of light at one or more wavelengths across a range of tested wavelengths. The method includes determining, using a discriminability module, a person's discriminability between light across the range of tested wavelengths. The method includes determining, using a transmission reduction module, a wavelength range to alter the color perception and/or color discriminability of the person based at least in part on the perception of light, the discriminability between light, and the one or more wavelengths. The method includes determining, using a node module, for each node of a plurality of nodes and based at least in part on the wavelength range, a parameter value, the parameter value comprising: a distance between the node and an adjacent node of the plurality of nodes, a shape of the node, a dimension of the node, a configuration of an arrangement of nodes of the plurality of nodes, a composition of a material coating a node, and/or a material composition of the node. The method includes performing at least one of the following: forming the node based at least in part on the shape of the node, the dimension of the node, the composition of the material coating the node, and/or the material composition of the node; forming a plurality of cavities in a substrate based at least in part on the distance between the node and the adjacent node of the plurality of nodes and/or the configuration of the arrangement of nodes of the plurality of nodes; and/or causing the plurality of nodes to be deposited onto a substrate based at least in part on the distance between the node and the adjacent node of the plurality of nodes and/or the configuration of the arrangement of nodes of the plurality of nodes.

In some examples, the substrate is separate from a body of a lens, and the method further includes transferring the plurality of nodes onto a side of a body of a lens.

In some examples, determining the person's discriminability between light across the range of tested wavelengths includes determining a sensitivity of cones in the person's retina to incident irradiation across the range of tested wavelengths. The range of tested wavelengths includes a range within the visible spectrum.

In some examples, the method includes determining the person's perception of light and the person's discriminability between light using an anomaloscope.

As described herein, a color vision deficiency (“CVD”) includes any deficiency in ability to see a color and/or discriminate between different colors. For example, CVD includes red-green color blindness, which manifests as a difficulty in seeing and/or discriminating between different shades of red, green, and/or yellow. CVD includes, for example, protanomaly and deuteranomaly.

As used herein, the term “CVD profile” includes at least one metric of an individual's CVD. A metric includes, for example, an ability for an individual to perceive and/or discriminate between colors in a certain wavelength range. A metric includes, for example, an ability of the individual to perceive light in a certain wavelength range and/or an ability of the individual to discriminate between light of different wavelengths within the wavelength range. In some examples, the CVD profile includes cone sensitivities across the visible spectrum of light.

A “transmission reduction profile” can be determined based at least in part on a CVD profile and/or on a targeted color discriminability and refers to a percentage of reduction of transmission of light in a certain wavelength range to alter a wearer's color perception and/or discriminability. In some examples, the wavelength range and/or percentage of reduction is selected to help restore a patient's color resolution and provide color vision near normal levels in at least one region of the visible spectrum. In other examples, the wavelength range and/or percentage of reduction is selected to alter perception of color differences (i.e., color discriminability) for a user with normal or near-normal vision. In some examples, the transmission reduction profile is determined to increase perception of color differences in a particular region of the visible light spectrum. In some examples, the transmission reduction profile is determined based at least in part on goal transmission reduction based on a particular application for a lens. In some examples, the application includes an application in which acute color discrimination in a particular region of the optical spectrum can help to improve performance. In various examples, the application includes: entertainment viewing, a sporting objective, performance in a recreational activity such as hunting, the practice of medicine, operating machines such as aircraft, welding, and/or a military application. In some examples, the goal transmission reduction is determined based at least in part a desired reduction of exposure to optical frequencies that may be harmful to a wearer and/or cause glare or vision damage. In some examples, the transmission reduction profile includes a percentage of transmission reduction and/or a wavelength range determined to help case migraine symptoms. In some examples, the percentage of reduction of transmission varies across the wavelength range. In some examples, the transmission reduction profile is transmission reduction as a function of wavelength of light.

The present disclosure includes methods and apparatuses to help alter a person's discriminability and/or perception of light through a customized lens based on an that person's deficiencies and/or color discriminability goals. Implementations of the present disclosure can help to improve color perception while minimizing alterations in chromatic and achromatic discriminability, which may affect depth and motion perception.

As used herein, the term “perception of color” include a person's perception of light at particular wavelengths. The terms “perception of light” and “perception of color” include perceptions of different shades of colors and perceptions of color differences. For example, a person's “perception of color” includes a person's perceived differences between two different displayed colors.

As used herein, the term “discriminability” refers to a person's ability to discriminate or differentiate between different colors, shades, and/or hues of light. “Color discriminability” includes a person's differentiation between light across the visible spectrum, including differentiation between light of different shades (e.g., different shades of green) and differentiation between light of different colors (e.g., differentiating between red and green).

Examples of the present disclosure include lenses configured to reduce transmission of light in certain wavelength ranges. Methods of the present disclosure include determining a wavelength range based on a CVD of an individual. For example, an anomaloscope is used to determine the individual's CVD, or wavelengths of light that the individual has difficulty perceiving and/or discriminating between. In some examples, the wavelength range is a range within a broader range of wavelengths of a color that the individual is deficient in and/or a range encompassing a boundary of a range of wavelengths of that color. For example, an individual experiencing deuteranomaly has difficulty discriminating between shades and perceiving light in the 490-570 nanometers (“nm”) range. To improve perception and discriminability for such an individual in certain portions of the visible spectrum, examples of the present disclosure include a lens for that CVD that blocks transmission of light around 560 nm, or approximately 550 to 580 nm.

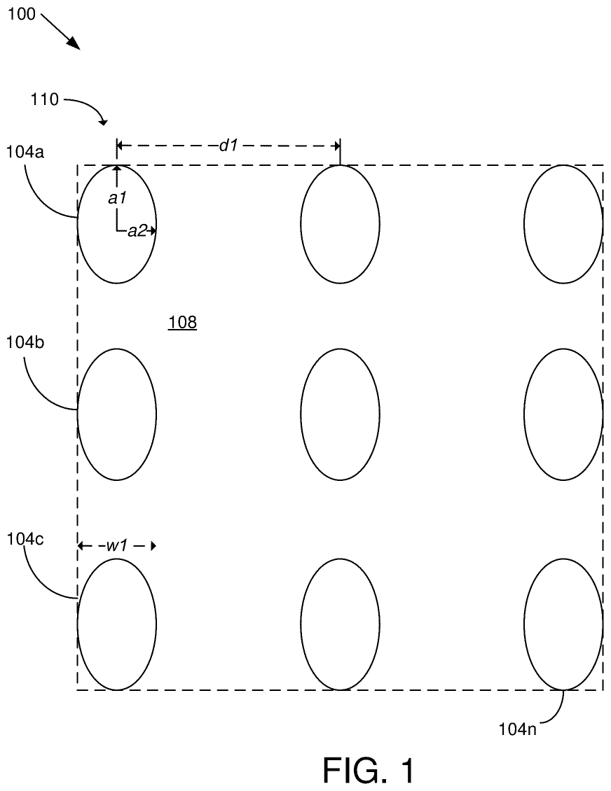

FIG. 1 illustrates a front view of a lens 100. In one or more examples, the lens 100 includes a convex side 108 of the lens 100 and a plurality of nodes 104a, . . . , 104n (“104”) formed onto the convex side 108.

In some examples, the lens 100 blocks or at least partially reduces transmission of light in a particular wavelength range for a wearer. In some examples, reducing transmission of light in a particular wavelength range helps to reduce confusion between adjacent colors on the visible spectrum. Creating customized wavelength gaps can help the better-functioning cones of a user's eye to differentiate between colors that may otherwise appear similar to the user.

In some examples, the lens 100 performs as a notch filter that is customized to help achieve a particular color discriminability goal. In some examples, the lens 100 is customized to help remedy CVD in persons who vary in classification and severity. In some examples, the degree to which the lens 100 helps with CVD varies based on the lighting conditions of a user's environment.

In some examples, one or more parameters of the nodes 104 is based at least in part on a transmission reduction profile of a person. In some examples, the values of the parameters include a value of: a distance d1 between a node 104a and an adjacent node 104b, an environment of the node 104, a shape of the node 104, a dimension of the node 104, a shape of an arrangement in which the nodes 104 are arranged on the convex side 108, and/or a material composition of the node 104. In some examples, the environment of the node includes at least one of: a material composition of the lens body 102, a composition of a material coating the node 104, and/or a composition of a material adjacent to the node 104 in the lens body 102.

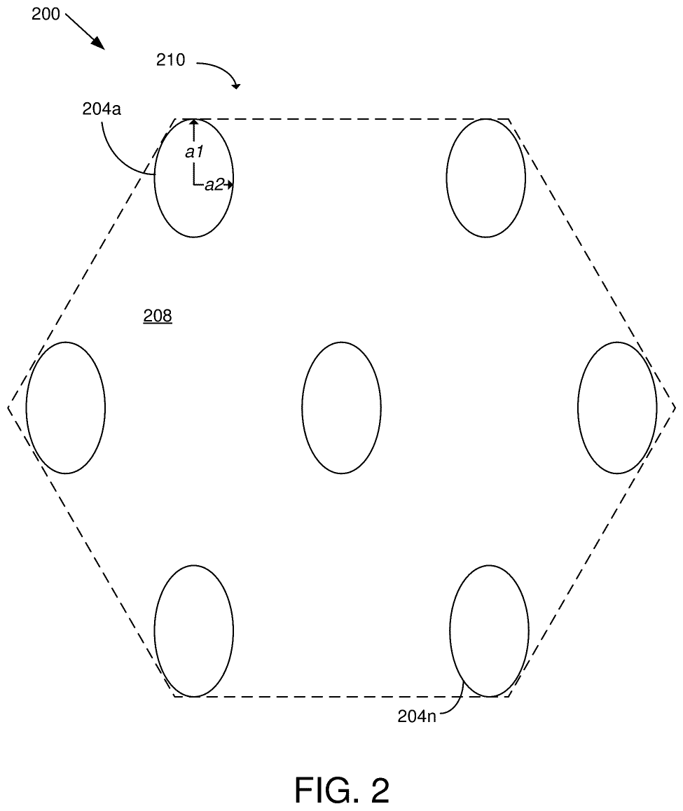

In some examples, the parameters are based at least in part on a transmission reduction profile. Parameters include, for example, composition of the nodes 104, dimensions a1 and a2 of the nodes 104, geometry of the nodes 104, environment surrounding the nodes 104, and/or spatial arrangement of the nodes 104. The composition of the nodes 104 includes, for example, materials of which the nodes 104 are made. The geometry of the nodes 104 includes, for example, at least one of a shape, and/or dimension of the node 104. In some examples, a dimension of the node 104 includes a size of the node 104, a volume of the node 104, a surface area of the node 104, a width w1 of the node 104, a major axis a1, a minor axis a2, and/or an aspect ratio (e.g., ratio of a major axis a1 to a minor axis a2) of the node 104. The spatial arrangement of the nodes 104 includes, for example, at least one of: a pitch (i.e., distance d1 between adjacent nodes 104) and arrangement of the nodes 104. For example, the arrangement of the nodes includes a configuration (e.g., shape) in which the nodes 104 are arranged, such as a rectangular (e.g., square) array 110. Arrangement of the nodes 104 also includes, in some examples, a hexagonal array 210, as shown in FIG. 2. In some examples, the customization of the lens 100 via the parameters of the nodes 104 replaces and/or helps to reduce infusion of the lens 100 with dyes and/or particles.

In some examples, the nodes 104 are regularly spaced. For example, the array 110 of nodes 104 includes rows of equally spaced nodes 104 and columns of equally spaced nodes 104. Each of the rows and columns are equally spaced with respect to adjacent rows and columns. In some examples, the array 110 of nodes 104 is a lattice of nodes 104. In some examples, the distance d1 between a node 104a and an adjacent node 104d and/or 104b is less than or equal to 1000 nm, or one micrometer (“μm”). For example, the distance d1 is not less than 10 nm and not greater than 1000 nm. In some examples, the distance d1 is not less than 400 nm and not greater than 1000 nm.

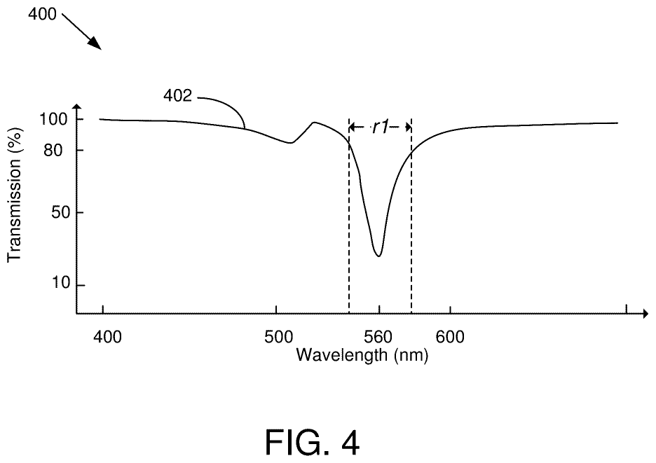

In some examples, the distance d1 between adjacent nodes 104 is based at least in part on (e.g., proportional to and/or approximately equal to) a wavelength within a wavelength range of light to reduce transmission of. In some examples, the distance d1 is a multiple of a wavelength within that wavelength range. Examples of the present disclosure include adjusting the distance d1 based at least in part on a wavelength of light to block transmission of. For example, to block transmission of light having wavelengths of approximately 560 nm, the distance d1 is set to approximately 560 nm and/or to a multiple of 560 nm (for example, 1120 nm). The transmission of light as a function of its wavelength for such a lens 100 is demonstrated by function 402 of FIG. 4.

In some examples, a dimension of the node 104 (e.g., a diameter and/or width w1) is based at least in part on the transmission reduction profile. In some examples, a dimension of a node 104 determined based on a transmission reduction profile includes the width w1. In some examples, the nodes 104 have a width w1 of less than 300 nm.

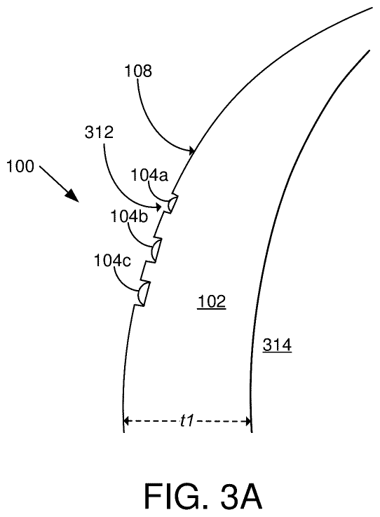

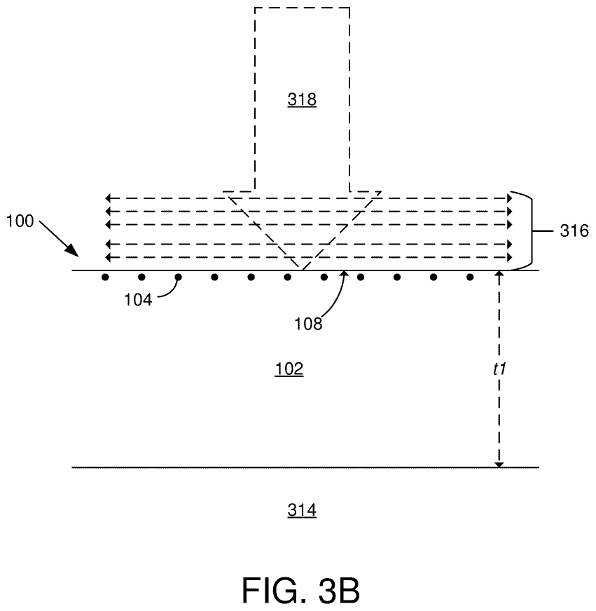

In some examples, as shown in FIG. 3A, the lens 100 includes a body 102 with a thickness t1. In some examples, the thickness t1 is based on a CVD profile. In other examples, the thickness t1 is independent of the CVD profile.

In some examples, the aspect ratio of the nodes 104 is determined based on a transmission reduction profile. In some examples, the aspect ratio of the nodes 104 is not less than 1 and not greater than 5. In some examples, the aspect ratio is not less than 3 and not greater than 5. For example, the aspect ratio is 1:4. For example, the length a2 of the minor axis of the node 104 is not less than 50 nm, and the length a1 of the major axis of the node 104 is not less than 150 nm. In some examples, the length a2 of the minor axis of th enode 104 is not less than 3 nm and not greater than 150 nm. In some examples, the length a1 of the major axis of the node 104 is not less than 3 nm and not greater than 150 nm.

Examples of the present disclosure include nodes 104 composed of, for example, at least one of the following materials: metal, ceramic materials, polymeric materials, graphene, graphene-like materials, oxide, dichalcogenide, aluminum, titanium dioxide, phosphorene, boron nitride, sililcene, black phosphorous, mXenes, stanine, perovskite, metallic-organic materials, topological insulators, semiconductors, semiconductor nanocrystals, quantum dots, or any combination thereof. For example, each of the nodes 104 is made of silver. In other examples, the nodes 104 include gold and/or copper. In other examples, the nodes 104 are composed of oxide and/or dichalcogenide. In some examples, the composition of the nodes 104 is homogeneous. In other examples, the nodes 104 are composed of two or more materials. In some examples, the nodes 104 include a first material and are coated with a second material that is different from the first material. In one or more examples, the second material is a polymeric material. In some examples, the nodes 104 are Janus particles, which include at least two different surfaces having different physical and/or chemical properties. In some examples, a parameter of the nodes 104 determined based on the transmission reduction profile includes whether the node 104 is composed of a single material or a mix of materials.

In some examples, the material is selected based at least in part on the transmission reduction profile. For example, as shown in FIG. 8, examples of the present disclosure include a lens 100 configured to reduce transmission of light around 560 nm. Such examples include nodes 104 composed of silver. However, other examples of the present disclosure include lenses 100 configured to reduce transmission of higher wavelengths of light to a lesser degree based on the transmission reduction profile of the person, and such examples include nodes 104 composed of gold and/or copper.

Although the nodes 104 shown in FIG. 1 are ellipse-shaped, examples of the present disclosure include other shapes of nodes 104. Such shapes include, for example, but are not limited to, shells, rings, spheres, semi-spheres, cylinders, ellipsoid, rods, cubes, and/or stars. In some examples, the nodes 104 are branched.

According to an example of the present disclosure, the lens 100 includes nodes 104 made of silver, shaped as ellipses, and having an aspect ratio of 4:1 (i.e., a length a1 of a major axis that four times the length a2 of the minor axis). In some examples, the lens 100 includes an array 110 of nodes 104 that is a square-shaped lattice with a pitch d1 of approximately 553 nm.

In some examples, a shape of the array 110 is based at least in part on the transmission reduction profile. For example, a square array 110, as shown in FIG. 1, may be selected based on a transmission reduction profile for a sharper, more tunable degree of transmission reduction. A hexagonal array 210, as shown in FIG. 2, may yield a lower degree of transmission reduction but may be preferrable for patients with less severe CVD. Another example of an array arrangement includes a triangular arrangement.

FIG. 3A illustrates a side, cross-sectional view of a lens 100, according to one or more examples of the present dislcosure. As shown in FIG. 3A, the lens includes a convex side 108 with nodes 104 formed onto a convex side 108. For example, the nodes 104 are formed into cavities 112 of the convex side 108. In some examples, the lens 100 is a contact lens fitted onto a cornea 314 of a person. In some examples, the surface area of the convex side 108 of the lens 100 is greater than or equal to one square centimeter (“cm²”). For example, the lens 100 has a diameter of 1.3-1.45 cm².

In some examples, the lens 100 has a thickness t1 of not less than 0.06 mm and not greater than 0.18 mm. In some examples, the percentage of transmission of light through the lens 100 is independent nearly independent of the thickness t1 of the lens 100. The thickness t1 refers to a thickness of the lens 100 at a thickest portion of the lens 100.

In some examples, the cavities 312 are arranged in an array 110 and/or lattice, with each cavity 312 receiving a single node 104. In some examples, the cavities 312 are arranged into an array 110 that can accommodate an arrangement of nodes 104 on the convex side 108 determined based at least in part on the CVD profile. In some examples, the cavities 312 are arranged in a square and/or rectangular array 110 to help facilitate arranging the nodes 104 in a square and/or rectangular array on the lens 100. In some examples, each cavity 312 receives a single node 104. In other examples, not all cavities 312 receive a node 104. In some examples, the nodes 104 are sealed into the cavities 312. For example, the nodes 104 are sealed into the cavities 312 with an overlay. An example of an overlay includes a biocompatible polymer.

Although FIG. 3A illustrates nodes 104 housed within cavities 312, examples of the present disclosure are not so limited. In some examples, the nodes 104 are embedded in the lens body 102. In some examples, the nodes 104 are introduced into the lens body 102 while the lens body 102 is being formed. In various examples, the nodes 104 are introduced into the lens body 102 after the lens body 102 is formed.

Although only three nodes 104a, 104b, and 104c are shown in FIG. 3A, examples of the present disclosure are not so limited. In some examples, the nodes 104a, 104b, and 104c shown in FIG. 3A are nodes of different rows of nodes 104. As discussed above, the nodes 104, in some examples, are spaced across a surface area of the covex side 108 of the lens 100. Hence, some examples include millions of nodes 104. Hence, although only nine nodes 104 are shown in FIG. 1 for simplicity, examples of the present disclosure are not so limited. In some examples, the lens 100 includes more than nine nodes 104.

FIG. 3B illustrates a cross-sectional view of the lens 100, perpendicular to the cross-sectional view of FIG. 3A. The lens 100 is positioned on a cornea 314 and includes uniformly spaced nodes 104. As shown in FIG. 3B, incident light 318 at a frequency of light that is scattered by regularly spaced nodes 104 is largely scattered perpendicular to the side 108 including the nodes 104, as shown by reflected light 316. In some examples, some of the scattered light is internally reflected in the lens body 102 itself between the convex surface 108 and the cornea 314. In one or more examples, the scattering of light 316 helps to reduce transmission to the cornea 314 and underlying cone photoreceptors distal to the corneal surface, at wavelengths at which the scattering of light by the nodes 104 becomes significant. As such, in some examples, the distance d1 between adjacent nodes 104 is approximately equal to a wavelength of a desired transmission reduction. For example, a lens 100 with nodes 104 spaced a distance d1 of approximately 560 nm apart can reduce transmission of light near a wavelength of 560 nm to the cornea 414. In some examples, a lens 100 with nodes 104 spaced a distance d1 of approximately 560 nm to target light near a wavelength of 560 nm includes a lens 100 with nodes 104 spaced a distance d1 of not less than 523 nm and not greater than 583 nm apart.

Although FIGS. 3A-B show the nodes 104 integrated into the outer convex side 108 of the lens 100, examples of the present disclosure are not so limited. In some examples, the nodes 104 are positioned on an inner, concave surface or within a body 102 of the lens itself. However, locating the nodes 104 on the outer convex side 108 helps to minimize potential for contact with the cornea 314 as well as minimize possibility of optical aberration within the body of the lens 100.

In some examples, the body 102 of the lens 100 is a soft lens made by injection molding. In various examples, the body 102 of the lens includes: polydimethylsiloxane (PDMS), a hydrogel, a hydroxyethyl methacrylate copolymer, methyl methacrylate, vinyl pyrrolidone, ethylene oxide, ethyl methacrylate, glyceryl methacrylate, polyethylene glycol methacrylate, polyvinyl alcohol, a silicone hydrogel material, Balafilcon A, Lotrafilcon B, fluorosilicone acrylate, and/or a rigid, gas-permeable material, such as copolymers of fluoropolymers and/or silicone acrylates. In some examples, a material of the lens body 102 is selected to help achieve a desired transmission reduction, based at least in part on the transmission reduction profile. In some examples, the parameter values selected based on the transmission reduction profile include a composition of a lens body 102, at least in regions within 700 nm of a center of a node 104. In some examples, the lens 100 has a diameter of not less than 1.3 and not greater than 1.45 cm.

FIG. 4 depicts a graph 400 of a transmission (%) vs. wavelength (“nm”) function 402 for a lens (e.g., lens 100, 200) of the present disclosure. In some examples, the function 402 is a representation of a transmission reduction profile for a wearer. As shown in FIG. 1, in some examples, the lens 100 decreases transmission of light in the wavelengths within a range r/to as low as below 40% at a wavelength of 560 nm. For example, the range r1 is approximately 545-575 nm. For wavelengths outside of the range r1, transmission remains at or above 70%. Examples of the present disclosure include determining the range r1 based at least in part on a CVD profile and/or desired alteration in color discriminability.

Examples of the present disclosure include a lens 100, 200 and/or lens system 300 that transmits a percentage of light % T at a given wavelength (λ) as defined by the following formula:

$$\% T ( λ ) = {10}^{2 - {C}_{ext} ( λ )} ,$$

where the coefficient of extinction, Cₑₓₜ (λ)=4πIm[1/(α(λ)⁻¹−S(λ))], results from the imaginary part of the inverse difference between reciprocal of polarizability, α(λ), and the sum, S(λ), of dipole interactions. Examples of the present disclosure include adjusting the polarizability, α(λ), by varying a parameter of a node 104. As such, examples of the present disclosure include adjusting parameters of nodes 104 to adjust the percentage of light transmitted at a given wavelength, based on a transmission reduction profile for an intended wearer. Examples of the present disclosure include adjusting the dipole sum, S(λ), by adjusting the arrangement of nodes, including, for example, the pitch (inter-node spacing d1). In some examples, the node parameters include: (1) at least one of a composition or geometry of the node 104 selected to adjust the percentage of light transmitted in a wavelength range, (2) an arrangement of the nodes 104 and/or distance d1 between nodes 104 selected to adjust the wavelength range for transmission reduction, and/or (3) environment of the node, which, in some examples, includes a material composition of the lens body 102 and/or a composition of a material that coats and/or is adjacent to the node 104.

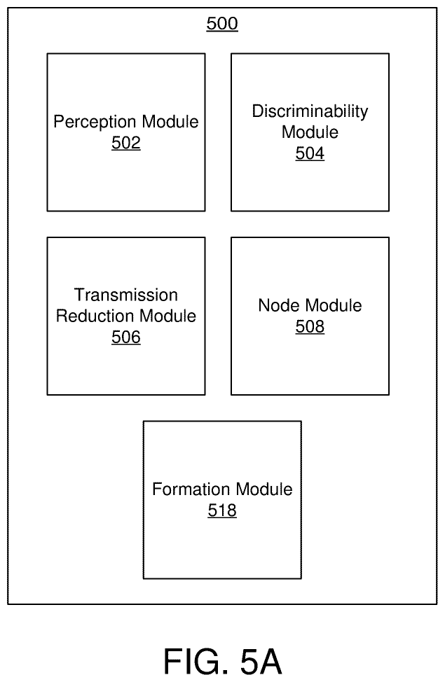

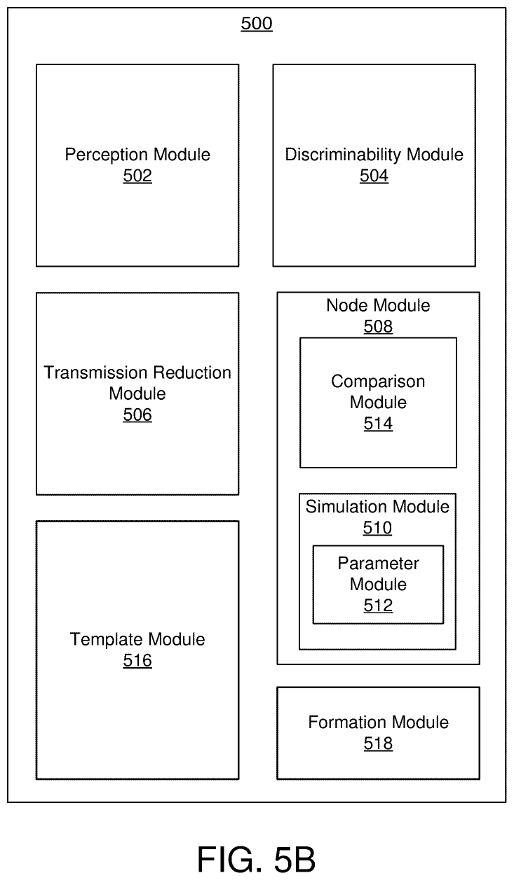

FIG. 5A illustrates an apparatus 500 for alteration of color discriminability, according to one or more examples of the present disclosure. The apparatus 500 includes a perception module 502, a discriminability module 504, a transmission reduction module 506, a node module 508, and a formation module 518. In some examples, all or a portion of the apparatus 500 is implemented with hardware circuits. In other examples, all or a portion of the apparatus 500 is implemented using a programmable hardware device. In other examples, all or a portion of the apparatus 500 is implemented with executable code stored on computer readable storage media where the code is executable by a processor.

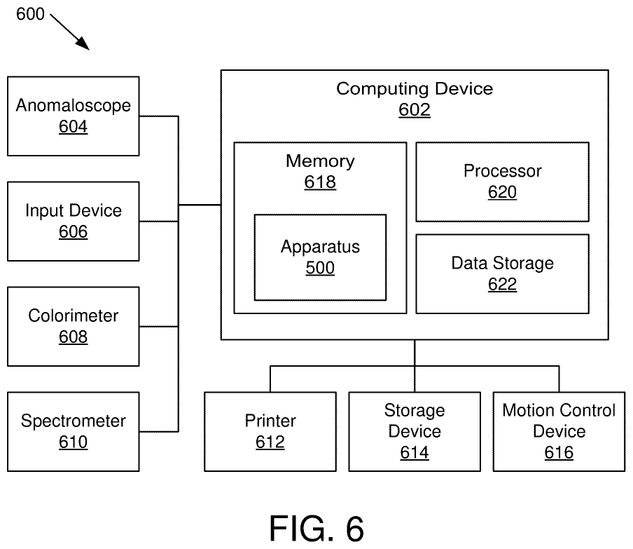

The apparatus 500 includes a perception module 502 configured to determine a person's perception of color. In some examples, the perception module 502 is configured to determine a person's perception of light from a stimulus (e.g., a light source and/or object that reflects light) at one or more wavelengths across a wavelength of tested wavelengths. For example, the perception module 502 instructs and/or receives results from an anomaloscope (e.g., anomaloscope 604 shown in FIG. 6) and/or another instrumental component of a psychophysical test involving color matching and colorimetry. Examples of the present disclosure include using a number of different techniques and instruments in color matching, such as colorimeters (e.g., colorimeter 608 shown in FIG. 6) or spectrophotometers (e.g., spectrometer 610 shown in FIG. 6). In some examples, the perception module 502 is configured to determine the person's perception of color using a Farnsworth-Munsell 100 test, a Farnsworth D-15 Test, a Cambridge Colour test, an Ishihara color vision test (e.g., using Ishihara plates), and/or color arrangement.

For example, for a patient experiencing deuteranomaly, the perception module 502 is configured to determine perceived color difference (ΔE′) based on cone sensitivities using a uniform color space (UCS) model. An example model of the present disclosure describes perceived color difference (ΔE) as follows:

$$Δ {E}^{′} = \sqrt{Δ {J}^{′ 2} + Δ {a}^{′2} + Δ {b}^{′2}} ,$$

Where a′ and b′ are functions of a person's perception of hue (h), as follows:

a′=M′ cos(h); and

b′=M′ sin(h).

M′ is a function of a person's perception of colorfulness (M), as follows:

$${M}^{′} = \frac{ln ( 1 + 0.0228 M )}{0.0228} .$$

J′ is a function of a person's perception of lightness (J), as follows:

$${J}^{′} = \frac{1.7 J}{1 + 0.007 J} .$$

As such, the perception module 502 determines perceived color difference (ΔE′) based at least in part on a person's perception of hue (h), colorfulness (M), and lightness (J). For example, the perception module 502 is configured to use results from an anomaloscope to determine the hue (h), colorfulness (M) and lightness (J) of a person's color perception in a tested wavelength range.

The apparatus 500 includes a discriminability module 504 configured to determine a person's discriminability between light of the one or more wavelengths across the range of test wavelengths. In some examples, the discriminability module 504 is configured to determine a person's discrimination between light of various shades, hues, and/or colors. In such examples, the light is emitted from a stimulus (e.g., a light source or object reflecting light). The discriminability module 504 is configured to instruct and/or receive results from an anomaloscope 604 and/or components of other psychophysical tests as explained above. In some examples, the discriminability module 504 is configured to determine color discriminability (ΔS′), or a test subject's ability to distinguish between different colors that may be problematic for persons with CVD, based on cone sensitivities using a receptor noise limited (RNL) model. An example model of the present disclosure describes perceived color discriminability (ΔS) as follows:

$$Δ S = \sqrt{\frac{{e}_{8}^{2} ( Δ {f}_{L} - Δ {f}_{M} ) + {e}_{M}^{2} ( Δ {f}_{L} - Δ {f}_{s} ) + {e}_{L}^{2} ( Δ {f}_{M} - Δ {f}_{s} )}{{( {ε}_{L} {ε}_{M} )}^{2} + {( {ε}_{L} {ε}_{S} )}^{2} + {( {ε}_{M} {ε}_{S} )}^{2}}} ,$$

where eᵢ represents the noise of each photoreceptor channel i=S,M,L. The cone receptor signal fᵢ for i=S,M,L is described as follows:

$$Δ {f}_{i} = ln \frac{{q}_{i}^{G}}{{q}_{i}^{0}} .$$

The receptor quantum catch qᵢ^{j} for receptor i=S,M,L under stimulus j is described as follows:

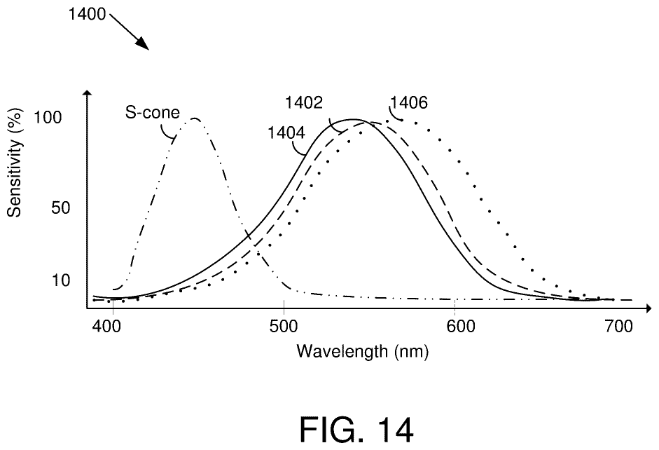

qᵢ^{j}=kᵢ∫I(λ)R^{j}(λ)T(λ)Sᵢ(λ)dλ,

where kᵢ is a normalization constant for receptor i. I(λ) is the spectral distribution of illumination incident on the stimulus (e.g., a color displayed an Ishihara plate), R^{j}(λ) is the normalized reflectance spectrum of stimulus j, where j=green (G) or orange (O) in an Ishihara plate, for example. T(λ) represents the normalized transmittance spectrum of the lens 100 itself. Sᵢ(λ) represents the normalized spectral sensitivity of receptor i, and λ represents the wavelength of illumination. As a patient's conc sensitivity S′M(λ) changes from mild to severe, the transmission reduction profile, or wavelength of transmission reduction (T(λ)) of the lens 100, should be adjusted to help optimize discriminability (ΔS′). In some examples, the discriminability module 504 is configured to obtain a CVD patient's cone sensitivity S′M(λ) (e.g., 1402 of FIG. 14). In some examples, the discriminability module 504 obtains the cone sensitivity through an anomaloscope 604. As shown in FIG. 14, a deuteranomalous patient's cone sensitivity S′_{M} (λ) 1402 is red-shifted from normal medium (M) cone sensitivity (S_{M} (λ), 1404), thus increasing the overlap with long (L) wavelength cone reception 1406, resulting in an increase in red-green color confusion for the patient.

In some examples, the transmission reduction module 506 and/or node module 508 is configured to classify the CVD based on the λ of the patient's derivation from normal (e.g., the patient's red-shift). In some examples, the λ of derivation includes a difference between the normal medium (M) cone sensitivity 1404 and the deuteranomalous patient's cone sensitivity 1402. In some examples, the discriminability module 504 is configured to classify the deuteranomalous patient's cone sensitivity 1402 as “mild”, “moderate”, or “severe.” In some examples, this classification is part of a CVD profile. In some examples, the direction of the shift (e.g., red-shift) is also part of the CVD profile. In some examples, the discriminability module 504 is configured to classify a cone sensitivity of ≤6 nm as “mild.” In some examples, the discriminability module 504 is configured to classify a cone sensitivity of greater than 6 nm and less than 18 nm as “moderate.” In some examples, the discriminability module 504 is configured to classify a cone sensitivity of greater than 18 nm as “severe.”

In some examples, the perception module 502 is configured to determine hue (h), colorfulness (M), and lightness (J) of the UCS model from illuminant power distribution, I(λ), spectral reflectance Rj(λ) of object j, and color matching functions. In some examples, the discriminability module 504 is configured to determine the receptor quantum catch (qij) for i=short(S) (1408), medium (M) (1404), and long (L) (1406) wavelength cones of the RNL model from, I(λ), Rj(λ), Si(λ), and transmittance (T(λ)) of ocular media, macular pigment, and/or a lens of the patient's eye. Receptor quantum catch (qij) includes, in some examples, the efficiency of a photoreceptor cell in capturing photons of a particular wavelength.

The apparatus 500 includes a transmission reduction module 506 configured to determine a transmission reduction profile, or a wavelength range, based at least in part on the perception of color, the discriminability between colors, and the one or more tested wavelengths. In some examples, the transmission reduction module 506 is configured to determine a percentage of transmission reduction of light within the wavelength range. In some examples, the transmission reduction module 506 is configured to determine the transmission reduction profile based at least in part on a desired alteration in color perception and/or discriminability. In some examples, the alteration is based at least in part on a CVD of the patient, and the transmission reduction module 506 is configured to determine a transmission reduction profile to improve the patient's CVD. In other examples, the alteration is based at least in part on another goal for a lens wearer, such as changing a user's discriminability between colors for an application, such as hunting.

In some examples, the transmission reduction module 506 is configured to update and/or create a CVD profile. In some examples, the transmission reduction module 506 is configured to determine a transmission reduction based on the CVD profile for a patient. For example, the transmission reduction module 506 determines a transmission reduction of a threshold percentage in a wavelength range of 540 nm to 560 nm for a patient experiencing low red-green color discriminability. In some examples, the transmission reduction module 506 outputs a function of optimal transmission reduction at various wavelengths, which could resemble function 402 of FIG. 4.

In some examples, the transmission reduction module 506 is configured to determine wavelength shifts across green and red portions of the visible light spectrum to help restore the patient's color resolution and provide color vision near normal. In some examples, the transmission reduction module 506 determines the transmission spectrum, or transmission reduction profile, able to provide the spectral wavelength shifts to improve vision in certain portions of the visible spectrum based on the wavelength shifts. In some examples, the transmission reduction mode 506 is configured to use data from both the perception module 502 and the discriminability module 504 to determine a transmission reduction profile.

The apparatus 500 includes a node module 508 configured to determine, for each node of a plurality of nodes 104 and/or for an entire plurality of nodes 104 to be formed onto a lens 100 and based at least in part on the wavelength range, a value of a parameter. In some examples, the node module 508 is configured to determine the value of the parameter based at least in part on the percentage of transmission reduction determined by the transmission reduction module 506. The parameter includes a distance d1 between a node 104a and an adjacent node 104b, a shape of the node 104, a dimension of the node 104, a shape of an arrangement in which the nodes 104 are arranged on the convex side 108, and/or a material composition of the node 104.

In some examples, the parameters also include an environment of the node 104. In some examples, the parameters include the optical environment of the node 104, or the refractive index of the material in the lens body 102 that surrounds the node 104. In some examples, the environment of the node includes at least one of: a material composition of the lens body 102 (e.g., PDMS, H20, air, and/or silica), a composition of a material in which the node 104 is coated, and/or a composition of a material adjacent to the node 104 on the lens body 102.

In some examples, the composition of the node includes materials adsorbed on surfaces of the node 104. In some examples, such materials include: molecules such as citrate, proteins, antibodies, nucleic acids; polymers such as polyvinylpyrrolidone (PVP), polyethylene glycol, poly(acrylic acid), polyvinyl alcohol, poly(N-vinylpyrrolidone), copolymers of PVP and vinyl acetate, polysaccharides, poly(sodium 4-styrenesulfonate), polydopamine, poly(ethyleneimine), and/or poly(N-isopropylacrylamide); surfactants such as cetyl-trimethlammonium bromide, Tween, sodium dodecyl sulfate, and/or shellac; silanes such as 3-aminopropyltriethoxysilane; chelators such as ethylenediaminetetraacetic acid and/or thiol ligands; ligands such as phospholipids and/or liposomes; and/or dendrimers, such as poly(amidoamine).

In some examples, the node module 508 uses both a UCS model and an RNL model to specify values of parameters. For example, the node module 508 is configured to determine, based on the transmission reduction module 506, to reduce transmission at around 560 nm to remedy deuteranomaly and uses both a UCS and RNL model to select values of parameters in order to achieve this reduction of transmission.

In some examples, the apparatus 500 includes a formation module 518 configured to perform at least one of the following: form a node 104 and/or cavity (e.g., cavities 312, 1312) in a substrate (e.g., lens body 102, template 1308, and/or mold 1502), and/or deposit a plurality of nodes 104 onto a substrate. At least one of the functions performed by the formation module 518 is performed based at least in part on a value of a parameter determined by the node module 508.

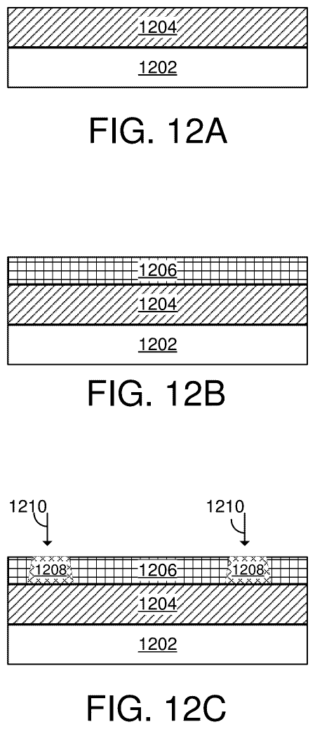

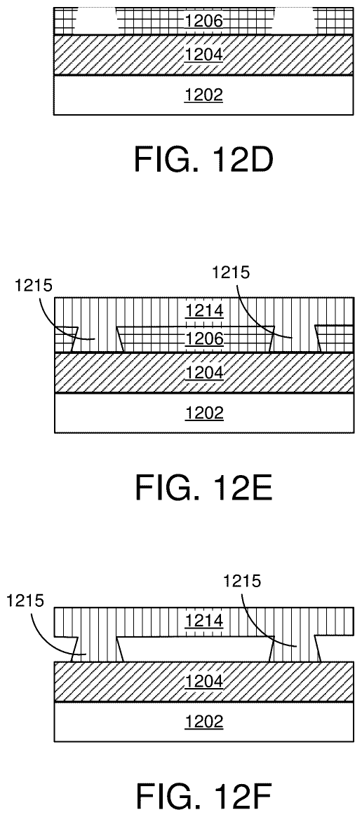

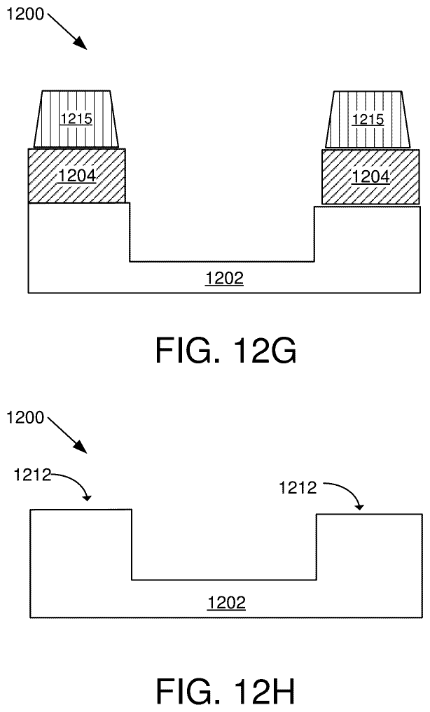

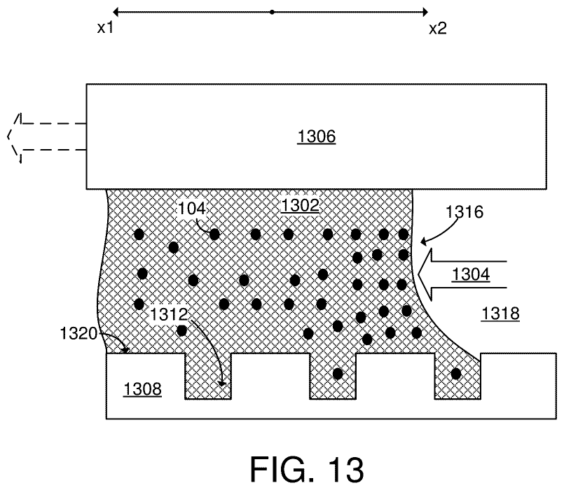

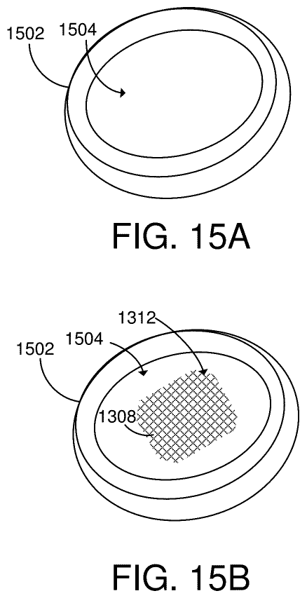

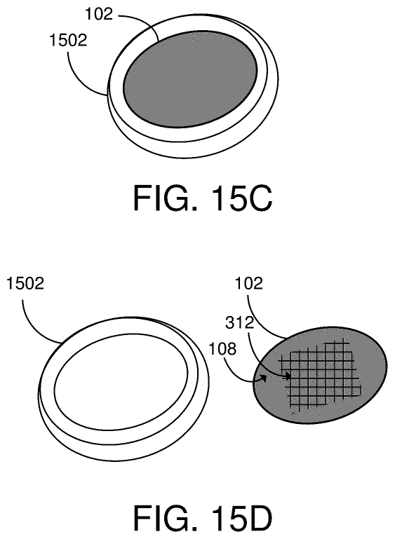

In some examples, the formation module 518 is configured to form a node 104 based at least in part on at least one of the following parameter values determined by the node module 508: node shape, node dimension, and/or material composition of the node 104. In some examples, the formation module 518 is configured to output a 3D file to allow the lens mold 1502 to be 3D-printed. In some examples, the formation module 518 is communicably coupled with a 3D printer, as shown in FIG. 6, to form a lens mold 1502 as shown in FIG. 15A and is configured to cause the 3D printer to incorporate a master stamp 1200 as shown in FIG. 12H in the lens mold to form cavities 1312 in the lens 1308 as shown in FIG. 13 according to the parameter values.

In some examples, the formation module 518 is configured to form a plurality of cavities in a substrate based at least in part on a value of at least one of the following parameters, as determined by the node module 508: the distance d1 between two adjacent nodes 104a and 104b of the plurality of nodes 104 and/or a shape of the arrangement 110 of nodes 104. A substrate includes at least one of: a contact lens body 102, a stamp 1200 for forming cavities 1312 in the template 1308 (as shown in FIGS. 12 and 13), and/or a mold 1502 for forming the contact lens body 102 (as shown in FIGS. 15A-D and 18). In some examples, forming cavities includes forming pillars 1212 in a stamp 1200 to be used to form cavities. In some examples, forming cavities includes forming cavities 1312 in a template 1308 to be transferred onto a lens body 102 and/or mold 1502. In some examples, forming cavities includes fabricating a lens body 102 and/or mold 1502 with cavities therein (e.g., cavities 312).

In some examples, the formation module 518 is configured to output a 3D file configured to be used to form cavities in the substrate. In some examples, the formation module 518 is communicably coupled with a 3D printer to fabricate the substrate, as shown in FIG. 6.

In some examples, the formation module 518 is configured to cause the plurality of nodes 104 to be deposited onto a substrate based at least in part on a value of at least one of the following parameters, as determined by the node module 508: the distance d1 between two adjacent nodes 104a and 104b of the plurality of nodes 104 and/or a shape of the arrangement 110 of nodes 104. In some examples, causing the plurality of nodes to be deposited onto the substrate includes causing the plurality of nodes 104 to be deposited into cavities (e.g., cavities 1312 and/or lens cavities 312).

In some examples, causing the plurality of nodes 104 to be deposited onto the substrate includes moving a superstrate to cause the plurality of nodes 104 to self-assemble onto the substrate via evaporative deposition. As shown in FIG. 6, in some examples, the formation module 518 is communicably coupled with a motion control device 616. In some examples, the motion control device 616 is configured to cause a superstrate 1306 to move and/or to control a velocity of the superstrate 1306.

FIG. 5B illustrates another apparatus 500 for alteration of color discriminability. The apparatus 500 includes a perception module 502, a discriminability module 504, a transmission reduction module 506, a node module 508, and a formation module 518 which are substantially similar to those described above in relation to the apparatus 500 of FIG. 5A. In various embodiments, the apparatus 500 of FIG. 5B includes a simulation module 510 with a parameter module 512 in the node module 508 and a comparison module 514 in the node module 508, and a template module 516, which are described below. In various embodiments, the apparatus 500 of FIG. 5A is implemented similarly to the apparatus 500 of FIG. 5A.

In some examples, the node module 508 includes a simulation module 510 configured to simulate lens-specific transmission reduction by spatially arranged optical nodes 104. In some examples, the lens simulation module simulates the behavior of a lens having nano-meter sized nodes (e.g., nodes 104) and a convex side 108 with a diameter of greater than or equal to one centimeter (“cm”). The simulation module 510 is configured to simulate node elements with varying parameters. The simulation module 510 is configured to generate a number of simulations of transmission of light through a lens, each simulation of the number of simulations having nodes of a test value of one or more test values of the parameter. The node module 508 uses these values to identify combinations that deliver a particular transmission spectrum (e.g., node parameters that deliver a transmission spectrum close to a transmission reduction profile).

The simulation module 510 is configured to bridge solutions to Maxwell's equations describing the activity of light at millimeter and nanometer scales. As such, the simulation module 510 simulates the transmission of light through a lens 100. In some examples, the simulation module 510 includes a parameter module 512, and the simulation module 510 bridges nanometer-scale node geometries across millimeter-scale network dimensions to link features selected by the parameter module 512 to spectral transmission profiles.

In some examples, the parameter module 512 is configured to vary certain parameters for the simulation. For example, the parameter module 512 instructs the simulation module 510 to test at least one of: distance d1 between nodes 104, individual node shapes, node dimensions (e.g., axis dimensions a1 and a2 and/or node size), shapes of node arrays 110, composition of nodes 104, and/or node environment.

In some examples, the node module 508 includes a comparison module 514. The comparison module 514 is configured to compare simulations conducted by the simulation module 510 to the transmission reduction determined by transmission reduction module 506. For example, the simulation module 510 outputs a function similar to the function 402 of FIG. 4 for a set of parameter values selected by the parameter module 512 (e.g., based on input from a user). The comparison module 514 compares the simulation to the transmission reduction profile by comparing a transmission reduction level within a wavelength range to a transmission reduction level for that wavelength determined by the transmission reduction module 506. For example, the transmission reduction module 506 determines a transmission reduction of 40% in the wavelength range of 540 to 580 nm. The comparison module 514 determines deviations from 40% transmission reduction in the critical wavelength range of 540 to 580 nm in each of the simulated lens architectures. In some examples, a “lens architecture” includes a set of values for a set of node parameters.

The node module 508, in some embodiments, is configured to select a lens architecture from one of the simulations. In some examples, the node module 508 determines, based on the comparison from the comparison module 514, which of the simulations is closest to the optimal transmission reduction in the critical wavelength range.

In some examples, the node module 508 determines, based on the simulations, that arrangement of nodes into a regular array, such as array 210 or rectangular array 110, induces collective optical scattering that constructively interferes at wavelengths at resonantly polarizable nodes. In some examples, the simulation module 510 is configured to demonstrate that such a parameter value produces a sharp decrease in transmission at particular wavelength(s). As such, in some examples, the node module 508 selects a lens architecture having regularly spaced nodes 104 in an array of hexagonal and/or square shape, according to the transmission reduction profile.

In some examples, the simulation module 510 determines that a lens architecture including nodes 104 comprising silver or gold spheres, silver or gold ellipses, and silver or gold shells about 60 nm in diameter greatly reduce transmission detectable by blue S-cones at 2.5-3.1 eV (400-500 nm). In some examples, the simulation module 510 is configured to convert from wavelength (λ) in nanometers to energy loss in electron volts (eV) using the relation: 1240/λ≈eV. In some examples, the node module 508 selects a lens architecture to help minimize transmission reduction outside of the critical wavelength range. For example, the node module 508 is less likely to select a lens architecture that greatly reduces transmission outside of the critical wavelength range. In some examples, the node module 508 excludes lens architectures comprising nodes 104 of gold or silver spheres, gold or silver ellipses, or gold or silver shells of 60 nm in diameter from consideration based at least in part on a transmission reduction profile not including reduced transmission in the 400-500 nm wavelength range.

In some examples, the apparatus 500 includes a template module 516 that translates the lens architecture determined/selected by the node module 508 to a two-dimensional template. In some examples, the template module 516 is configured to translate the node and/or lens architecture determined/selected by the node module 508 into a 3D file to be used to manufacture at least one of: a node 104, lens body 102, a stamp 1200, a template 1308, and/or a mold 1502.

FIG. 6 illustrates a system 600 for correction of color vision deficiency, according to one or more examples of the present disclosure. In some examples, the system 600 includes an apparatus 500, a computing device 602, an anomaloscope 604, an input device 606, a colorimeter 608, a spectrometer 610, a printer 612, a storage device 614, and/or a motion control device 616. In some examples, the apparatus 500 is substantially similar to the apparatuses 500 shown in FIGS. 5A-B.

In some examples, the computing device 602 includes memory 618 and a processor 620. In some examples, the processor 620 is configured to execute code stored in the memory 618. In some embodiments, the memory 604 is volatile memory, such as random access memory (“RAM”). In some examples, the memory 618 includes all or a portion of the apparatus 500, which may be loaded into the memory 604 as needed. In some examples, the computing device 602 includes data storage 622. The data storage 622 includes, for example, non-volatile storage such as a solid-state drive (SSD) and a hard disk drive (HDD), or the like. In various embodiments, the memory 618 and data storage 622 are computer readable storage media, which are non-transitory.

In some examples, the computing device 602 is communicably coupled to and configured receive input from and/or transfer data to at least one of the following: an anomaloscope 604, an input device 606, a colorimeter 608, and/or a spectrometer 610. In some examples, the perception module 502 is configured to determine a person's perception of light at one or more wavelengths across a range of tested wavelengths based at least in part on the input. In some examples, the discriminability module 504 is configured to determine the person's discriminability between light of the one or more wavelengths across the range of tested wavelengths based at least in part on the input.

In some examples, the anomaloscope 604 includes a number of light sources configured to emit light across a range of tested wavelengths. In some examples, the anomaloscope 604 also includes a number of dials configured to allow the user to input their perception of the emitted light. In some examples, a first light source emits light of a particular wavelength range, and the number of dials are configured to allow the user to adjust wavelengths of light emitted by a second light source. In some examples, the user stops adjusting the wavelengths of the second light source when the user perceives that the second light source is emitting a color that matches a color of the first light source. As such, input from the anomaloscope 604 includes, in some examples, discrepancy between the color actually being emitted by the second light source and the color that the user perceives to be matching the second light source (i.e., the color of light emitted by the first light source).

In some examples, the colorimeter 608 is configured to measure a wavelength of light reflected by a colored object. In some examples, the discriminability module 504 is configured to compare a person's perception of light (e.g., the color that the person perceives) to an actual wavelength of the light, as measured by the colorimeter 608. In some examples, the discriminability module 504 is configured to determine the person's discriminability between light of one or more wavelengths across the range of tested wavelengths based on this comparison.

In some examples, the spectrometer 610 is configured to emit and/or analyze light across the range of tested wavelengths. In some examples, the spectrometer 610 is configured to measure wavelengths of light in a similar manner as described above with respect to the colorimeter. In some examples, the spectrometer 610 is configured to emit and/or analyze light across the entire visible wavelength spectrum simultaneously.

In some examples, the input device 606 includes any device configured to provide input to the computing device 602. In some examples, the input includes: the CVD profile, the transmission reduction profile, a color perception, and/or color discriminability of the user. In some examples, the input includes test results from the anomaloscope 604, colorimeter 608, spectrometer 610, or the like. In some examples, the input includes test results form an Ishihara test.

In some examples, the computing device 602 and/or the apparatus 500 are communicably coupled with at least one of a printer 612, a storage device 614, and/or a motion control device 616. In some examples, the formation module 518 is configured to cause at least one of the printer 612, storage device 614, and/or motion control device 616 to perform a function.

In some examples, the printer 612 is a device configured to manufacture and/or assemble at least one of: a node 104, a stamp 1200, a template 1308, a lens body 102, and/or a mold 1502. In some examples, the printer 612 is a 3D printer. In some examples, the printer 612 is configured to manufacture a node 104 based at least in part on a value of a parameter determined by the node module 508. In some examples, the printer 612 is configured to print a template configured by the template module 516. In some examples, the printer 612 is configured to manufacture pillars and/or cavities in at least one of a stamp 1200, template 1308, lens body 102, and/or mold 1502 based at least in part on a value of a parameter determined by the node module 508.

In some embodiments, the storage device 614 is a portable non-volatile data storage device, such as a flash memory drive, a hard disk drive, or the like. In some examples, the storage device 614 includes an output file that includes code and/or data configured to be used to manufacture at least one of a node 104, a stamp 1200, a mold 1502, and/or a lens body 102 based on a parameter value determined by the apparatus 500. In some embodiments, the apparatus 500 transfers code and/or data in the form of an output file to the storage device 614 to be used to create a node 104, a stamp 1200, a mold 1502, a lens body 102, etc. In some examples, the output file is a 3D model, such as a computer-aided design file, an object file, and/or a stereolithography file. In some examples, the output file is a file to be used by the printer 612. In some examples, the output file output 614 is a program of the computing device 602 and/or a module of the apparatus 500.

In some examples, the motion control device 616 is a device configured to control deposition of nodes 104 into cavities 312, 1312. In some examples, the motion control device 616 is a device configured to control movement of a superstrate 1306, as shown in FIG. 13, causing evaporative deposition of nodes 104 into cavities 1312 of a template 1308. In some examples, the motion control device 616 comprises at least one of a robotic arm, a mechanical arm, and/or a linear actuator. In some examples, the motion control device 616 is communicably coupled to the apparatus 500 to move the superstrate 1306 at a velocity configured to achieve node deposition satisfying at least one of the parameter values.

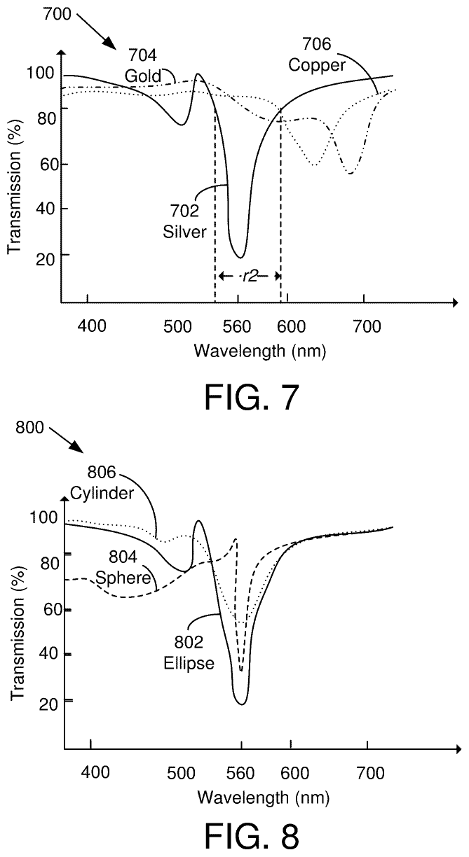

FIG. 7 illustrates a graph 700 of functions of transmission (%) vs. wavelength (nm) for nodes composed of each of: silver 702, gold 704, and copper 706. In some examples, the graph 700 is created by the simulation module 510. In some examples, the node module 508 selects (e.g., nodes 104) composed of materials selected to optimize transmission reduction within certain wavelength ranges. For example, the node module 508 selects nodes 104 made of silver to be compatible with a transmission reduction profile having a high degree of transmission reduction in a narrow wavelength range around 560 nm. For transmission reduction profiles indicating lower degrees of transmission reduction and at wider ranges of higher wavelengths, the node module 508 selects nodes 104 made of gold and/or copper.

FIG. 8 illustrates a graph 800 of functions of transmission (%) vs. wavelength (nm) for nodes having different shapes. Specifically, FIG. 8 illustrates transmission vs. wavelength for: ellipse shapes 802, spherical shapes 804, and cylindrical shapes 806. In some examples, the graph 800 is generated by the simulation module 510. As shown in FIG. 8, the node module 508 selects nodes having an ellipse shape for a transmission reduction profile around 560 nm with a transmission of less than 40%. In some examples, the selection is based at least in part on a comparison of the functions for ellipse shapes 802, spherical shapes 804, and cylindrical shapes 806 to the transmission reduction profile. In some examples, the transmission reduction profile is based at least in part on an estimated improvement in a person's CVD by reducing transmission at around 560 nm. In some examples, selection of the ellipse shape helps to achieve that improvement. In another example, the node module 508 selects nodes 104 having a cylindrical shape for a transmission reduction profile around 560 nm with less transmission reduction (e.g., for a patient with less severe CVD). In such examples, nodes 104 having a cylindrical shape may be preferable, since transmission reduction outside of the 540-570 nm is also less for the cylindrical shapes.

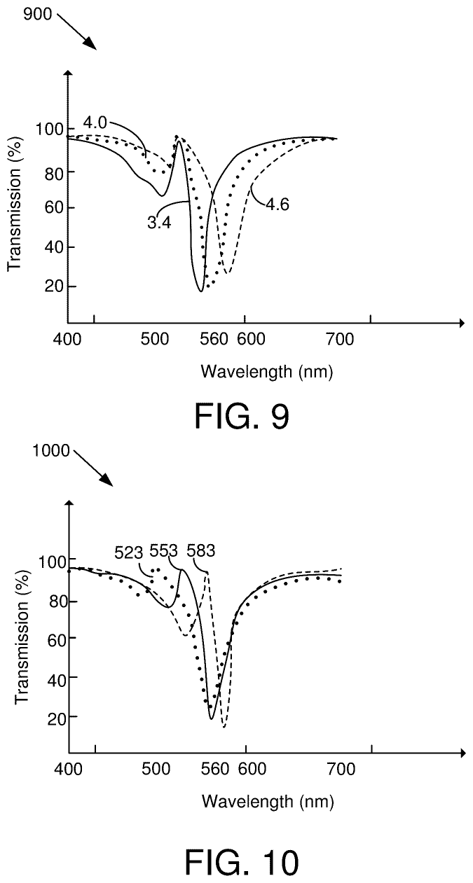

FIG. 9 illustrates a graph 900 of functions of transmission (%) vs. wavelength (nm) for nodes 104 having different aspect ratios. Specifically, FIG. 9 illustrates functions of transmission vs. wavelength for nodes 104 having aspect ratios of: 3.4, 4.0, and 4.6. In some examples, the node module 508 selects nodes 104 having an aspect ratio of 4.0 based at least in part on the transmission reduction profile including transmission reduction within a narrower range of wavelengths around 560 nm. In some examples, the node module 508 selects nodes 104 having an aspect ratio of 4.6 for a transmission reduction profile with transmission reduction at higher wavelengths (e.g., around 580 nm).

FIG. 10 illustrates a graph 1000 of functions of transmission (%) vs. wavelength (nm) for groups of nodes 104 having different pitch spacing. Specifically, FIG. 10 illustrates transmission vs. wavelength for nodes 104 of pitch spacing d1 of 523 nm, 553 nm, and 583 nm. In some examples, the node module 508 selects node architectures having pitch spacing d1 of 553 nm based on the transmission reduction profile including a wavelength range around 560 nm with a transmission of 20-40%. In such examples, the pitch spacing d1 of 553 nm, which is within and/or close to the wavelength range, helps to improve CVD by reducing transmission of light in the wavelength range. In another example, the node module 508 selects node architectures having a pitch spacing d1 of 583 nm based on the transmission reduction profile including a higher wavelength range and greater transmission reduction.

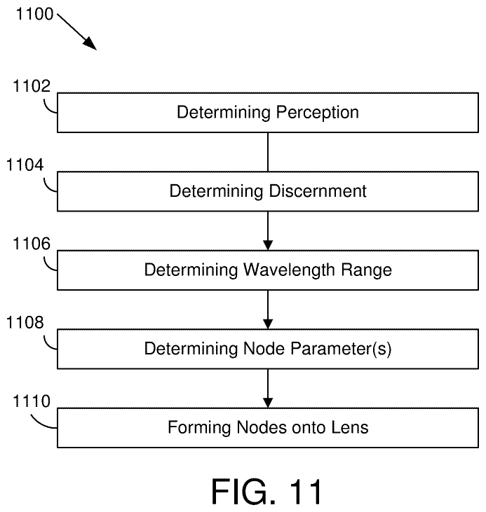

FIG. 11 illustrates a method 1100 of manufacturing a lens system 300. The method 1100 begins and determines 1102 a person's perception of color at one or more wavelengths across a range of tested wavelengths. The method 1100 includes determining 1104 a person's discriminability between colors, or discriminability between light of the one or more wavelengths across the range of tested wavelengths. The method 1100 includes determining 1106 a wavelength range based at least in part on the perception of color, the discriminability between colors, and the one or more wavelengths. The method 1100 includes determining 1108, for each node of a plurality of nodes and based at least in part on the wavelength range, a parameter value, the parameter value comprising: a distance between the node and an adjacent node of the plurality of nodes, a shape of the node, a dimension of the node, a shape of an arrangement in which the plurality of nodes are arranged on the side, a composition of a material coating the node, and/or a material composition of the node.

The method 1100 includes forming 1110 the plurality of nodes 104 onto a lens 100. In some examples, the method 1100 includes preparing a silicone master stamp (e.g., stamp 1200) consisting of pillars (e.g., pillars 1212) with which to nanoimprint a template with cavities (e.g., cavities 1312) into which self-assembling nanosized nodes are deposited. In some examples, the pillars 1212 are shaped cylindrically. In some examples, the method includes forming the template onto the lens. In other examples, the method includes forming the cavities directly into the lens itself and subsequently depositing the nodes into the lens cavities.

In some examples, the method 1100 includes forming nodes 104 by synthesizing silver ellipse nanoparticles to specifications selected by node module 508 according to a wet chemistry polyol method. In some examples, the node module 508 selects nodes of a silver composition based at least in part on the transmission reduction profile. In some examples, the method 1100 includes adding 0.5 mL of aqueous silver nitrate (1 M) and 2.5 mL of poly(vinyl pyrrolidone) (1 M, MW≈40 000) to 25 mL of poly(ethylene glycol) 600 solution in a flask with stirring. Examples of the present disclosure include obtaining a grey silver colloid (e.g., colloid solution 1302) after transferring the flask to an oil bath and heating the flask at 100 degrees Celsius for 6-10 hours (e.g., approximately 8 hours).

FIGS. 12A-H illustrate a method of forming a master stamp 1200 for forming a plurality of cavities in a lens body 102 of a lens 100 and/or in a template to be formed onto the lens 100. The master stamp 1200 includes pillars 1212 with which to imprint a lattice of cavities (e.g., cavities 1312 of FIG. 13, cavities 312 of FIG. 3A) onto a template (e.g., template 1308 of FIG. 13) into which nodes 104 self-assemble. In some examples, the template 1308 is a portion of the lens body 102. In other examples, the template 1308 is originally separate from the lens body 102 but is later formed onto the lens body 102 to become part of the lens 100.

As shown in FIG. 12A, in some examples, forming the stamp 1200 includes forming a silicon substrate 1202 of the stamp 1200 and forming a resist 1204 onto the silicon substrate. In some examples, the resist 1204 is conductive indium tin oxide (“ITO”). In some examples, the method includes spin coating another resist 1206, such as polymethylmethacrylate (PMMA), onto the ITO 1204, as shown in FIG. 12B.

As shown in FIG. 12C, methods of the present disclosure include forming, via an electron beam 1210, a plurality of pillars 1208 on the resist 1204 and into the additional resist 1206 with dimensions corresponding to dimensions of pillars 1212 of the stamp 1200 with which to imprint cavities 1312 into a template 1308 to contain a node 104. In some examples, the method includes patterning desired dimensions for the pillars 1208 into the resist using the electron beam(s) 1210, which desired dimensions correspond to desired dimensions for the cavities. As shown in FIG. 12D, the method includes removing the pillars 1208 from the additional resist.

As shown in FIG. 12E, in some examples, the method includes forming a metallic material 1214 onto the additional resist 1206, thus forming pillars metallic material pillars 1215 in the metallic material 1214. For example, the method includes plating the metallic material onto the additional resist 1206. As shown in FIG. 12F, the method includes removing portions of the additional resist 1206 between each of the plurality of metallic material pillars 1215. As shown in FIG. 12G, the method includes etching a portion of the substrate 1202. For example, the method includes cryogenically etching the substrate 1202 with oxygen and SF₆.

As shown in FIG. 12H, the method includes removing the metallic material 1214 and the metallic material pillars 1215 from the resist 1206 by etching the metallic material 1214 and its metallic material pillars 1215. For example, the method includes removing the metallic material 1214 through reactive ion etching. In some examples, the resulting master stamp 1200 includes pillars 1212 with dimensions that correspond to desired cavity dimensions for a template 1308. In some examples, the desired cavity dimensions are relative differential, δ/a≤0.1 for the cavity minor axis radius (a), δ/d≤0.1 for the cavity depth (d), and δ/b≤0.1 for the cavity major axis radius (b). This corresponds to cavity dimensions of 2a+δ (minor and depth) and 2+δ (major), respectively, within which the node 104 deposits upon self-assembly.

In some examples, the stamp 1200 is adaptable to customize dimensions of the node cavity 1312 size for different node 104 sizes. In some examples, this is enabled by coating it with a layer of etchable ITO or metal. In some examples, the pillar 1212 size is adjustable via methods of electro-less plating thin metal layers.

After the master stamp 1200 is formed, methods of the present disclosure include using the stamp 1200 to form a template 1308 for forming the lens 100. In some examples, the template 1308 is a lithographed template made by forming a polymeric film over the stamp (e.g., stamp 1200). Such examples include introducing a polymeric material such as polydimethylsiloxane to the surface of the stamp 1200. The polymeric material then conforms around the pillars 1212 and hardens into a film. Some examples include removing the film from the stamp 1200 to yield a template 1308. In some examples, the template 1308 is formed as a part of the lens body 102 itself. In some examples, the template is inserted into the lens body 102. Other examples include thermally melding the template 1308 to an inner cavity of a mold (e.g., a concave surface 1504 of a mold 1502) after softening the inner cavity by heat, forming a patterned cavity mold. Some examples include forming the lens 100 via the mold and transferring the nodes 104 from the mold into cavities 312 in the lens body 102.

Methods of the present disclosure include causing nodes 104 to self-assemble into the template 1308. In some examples, methods include self-assembling the nodes 104 into cavities 1312 in the template 1308 by evaporative deposition of a moving colloid solution 1302 via a wavefront 1304. FIG. 13 illustrates a method and a system 1300 for assembling nodes 104 into cavities 1312 of the template 1308.

FIG. 13 illustrates a method of self-assembling nodes 104 into cavities 1312 via evaporative deposition. In some examples, the cavities 1312 are cavities of a template 1308. In examples in which the template 1308 is formed onto the lens 100, the cavities 1312 are also cavities 312 of the lens body 102. The method includes sandwiching a solution 1302 of nodes 104 between the template 1308 and a moving superstrate 1306. In some examples, the template 1308 is positioned below the solution 1302, and the moving superstrate 1306 is positioned above the solution 1302, such that the nodes 104 are deposited into the cavities 1312 in the template 1308.

Some examples of the present disclosure include selecting a distance between the superstrate 1306 and the template 1308 such that the solution 1302 adheres to the superstrate 1306 via surface tension. In some examples, the solution 1302 moves with the superstrate 1306. In some examples, velocity of the wavefront 1304 and corresponding settling rate of the nodes 104 into cavities 1312 is governed by the velocity of the superstrate 1306. In some examples, the method includes moving the superstrate 1306 at a velocity of approximately the rate of evaporation. In some examples, evaporation of the solution 1302 creates a current that pulls the nodes 104 towards an interface 1316 between the solution 1302 and surrounding air 1318. As such, as shown in FIG. 13, as the superstrate 1306 moves in a direction x1, the nodes 104 moves in an opposite direction x2. In some examples, the nodes 104 become more concentrated, which causes the nodes 104 to move via Fickian diffusion into less concentrated spaces, such as the cavities 1312.

Some examples include selecting and controlling at least one parameter such that each node 104 is deposited into a cavity 1312 and only a single node 104 is deposited into each cavity 1312. Such parameters include, for example, cavity 1312 dimensions (e.g., cavity size), node 104 dimensions (e.g., node size), velocity of superstrate 1306 motion, ambient conditions such as temperature or humidity, and/or any combination thereof. In some examples, a non-cavity surface 1320 of the template 1308 includes a hydrophobic material. In some examples, one or more of the nodes 104 are coated with a hydrophilic coating. This coating can help to prevent the nodes 104 from adhering to the non-cavity surface 1320.

In some examples, the solution 1302 is a colloid solution. In some examples, the superstrate 1306 is made of a silicon dioxide material. In some examples, the superstrate 1306 is made of glass. In some examples, the superstrate 1306 is made of a silicate material, such as mica. In some examples, the superstrate 1306 is a mold 1502, and the mold 1502 is controlled to help encourage nodes 104 to assemble into cavities 312 in the lens body 102.

FIGS. 15A-E further illustrate a method of forming the lens 100. Some examples include forming the lens 100 via injection molding. Examples include a mold 1502 in which the lens body 102 is formed. In some examples, the method includes forming the mold 1502 based at least in part on a lens architecture for the lens 100. In some examples, forming the mold 1502 includes 3D-printing the mold 1502. In some examples, the mold 1502 is made of a polypropylene material.

As shown in FIG. 15A, in some examples, the mold 1502 includes a concave surface 1504. In some examples, the method includes forming the lens body 102 onto the concave surface 1504. As shown in FIG. 15B, some examples include forming the template 1308 onto the concave surface 1504 to create cavities 1312 in the concave surface 1504. In other examples, cavities are formed directly onto the concave surface 1504 (for example, using the stamp 1200). In some examples, the mold 1502 is 3D-printed with a substrate containing cavities formed in the concave surface 1504. Although FIG. 15B shows a rectangular template 1308, examples of the present disclosure are not so limited, and the template 1308, in some examples, is shaped differently and/or covers a higher percentage of the area of the concave surface 1504 than is shown in FIG. 15B.

In some examples, the nodes 104 are already assembled into the cavities 1312 of the template 1308 when the template 1308 is formed onto the mold 1502. In other examples, the method includes assembling the nodes 104 into the cavities 1312 after forming the template 1308 onto the mold 1502. For example, the method includes assembling the nodes 104 into cavities 1312 on the mold 1502 via the method illustrated in FIG. 13. In various examples, the method includes forming the lens body 102 using the mold 1502 without depositing the nodes 104 into the mold 1502 and subsequently depositing the nodes 104 into lens body 102 cavities 312 using evaporative deposition, as shown in FIG. 13, and/or transfer printing.

As shown in FIG. 15C, in some examples, the method includes depositing material onto the concave surface 1504. In some examples, the method includes heating, curing, solidifying, and/or cooling the material to form the lens body 102. In some examples, the lens body 102 includes cavities that are a mirror image of the cavities formed in the mold 1502 or of pillars formed in the master 1200 (e.g., cavities 312 are a mirror image of pillars 1212). In some examples, the lens body 102 includes cavities 312 on a convex surface 108 of the lens body 102. In some examples, the template 1308 includes template cavities 1312 that face the concave surface 1504, and the method includes forming lens body 102 onto the template 1308 while the template 1308 is within the mold 1502 such that the cavities 1312 of the template 1308 become cavities 312 of the lens body 102.

As shown in FIG. 15D, in some examples, the method includes removing the lens body 102 from the mold 1502. In some examples, removing the lens body 102 from the mold 1502 yields a lens body 102 with an array of cavities 312 on the convex surface 108. In some examples, removing the lens body 102 from the mold 1502 includes removing the template 1308 from the mold 1502. Some examples of the present disclosure include solidifying the lens body 102 by free-radical polymerization while shaping it with centripetal force.

Examples of the present disclosure include transferring nodes 104 from one surface to another via transfer printing. Some examples include transferring nodes 104 from cavities in the mold 1502 to cavities 312 in the lens body 102. Some examples include transferring nodes 104 into cavities 1312 of the template 1308. Some examples include transferring nodes 104 from the cavities 1312 of the template 1308 onto the lens body 102. Some examples include transferring nodes 104 from the cavities 1312 of the template 1308 onto the mold 1502.

In some examples, transfer printing the nodes 104 includes transfer printing via laser induction. In some examples, a method of transfer printing includes depositing the nodes 104 into the template 1308's cavities 1312 (e.g., via evaporative deposition, as shown in FIG. 13) and subsequently inverting the template 1308 onto the lens body 102. In some examples, inverting the template 1308 onto the lens body 102 includes positioning the template 1308 such that cavities 1312 of the template 1308 are substantially aligned with cavities 312 in the lens body 102. In various examples, the method includes placing weight (e.g., weights of less than 10 grams) onto the template 1308 to help promote contact between the template 1308 and the lens body 102. In some examples, the method includes irradiating the template 1308 and lens body 102 with a laser and removing the template 1308 from the lens body 102, leaving the nodes 104 on the lens body 102 (e.g., within the lens cavities 312). In some examples, irradiating the template 1308 and lens body 102 with the laser involves irradiating for a period of about 10 minutes, or between and inclusive of 5 and 15 minutes. In some examples, the laser emits light with a wavelength of between and inclusive of 500 and 550 nm. In some examples, the wavelength is approximately 532 nm. In some examples, the intensity of the light is approximately 50 milliwatts (“mW”). In some examples, irradiating the template 1308 and lens body 102 includes irradiating an interface between the template 1308 and the lens body 102 by periodically interrupting the emitted light (e.g., using an optical chopper). In some examples, the frequency of interruption is approximately 6000 Hz.

In some examples, transfer printing the nodes 104 includes transfer printing using resinous adhesion via a resin, such as shellac. In some examples, the resin is Bulls Eye® Shellac. In some examples, the method includes coating a surface with the resin and inverting the template 1308 containing the nodes 104 onto the coated surface. In some examples, the method includes removing the template 1308 from the surface and curing the template 1308 such that the resin solidifies onto the template 1308. In some examples, the time period for curing is approximately 90 seconds. In some examples, the method also includes curing the template 1308 while the template 1308 is still inverted onto the surface. In some examples, the method includes inverting the template 1308 onto a lens body 102 after the resin has been cured to the template 1308. In some examples, the method includes applying pressure to the template 1308 for approximately 60 seconds. In some examples, the method includes removing the template 1308 from the lens body 102 such that a layer of the resin, to which the nodes 104 are adhered, remains on the lens body 102.

In some examples, after the lens body 102 is formed and the nodes 104 are deposited onto the lens body 102, the resulting lens 100 includes a lens body 102 with cavities 312 containing nodes 104, as shown in FIG. 3A. In some examples, the depth of a cavity 312 is substantially equal to a dimension of the node 104 which the cavity 312 is to receive. As referred to herein, “substantially equal to” means, for example within 10%.

Other examples of methods to assemble nodes 104 in regular arrays 110 include, for example: heat-induced coalescence of metal island films deposited in lithographed cavities on a ceramic substrate into nanonodes; colloidal self-assembly of silica particles to hexagonally mask a surface, followed by deposition of resist, silica particle removal, and metallization to form nanonodes; (iii) hole-by-hole removal of photoresist from a conductive surface by electron beam lithography followed by metallization; and (iv) self-assembly of nodes deposited into cavities formed by nanoimprint lithography from a silicon master onto a resist.

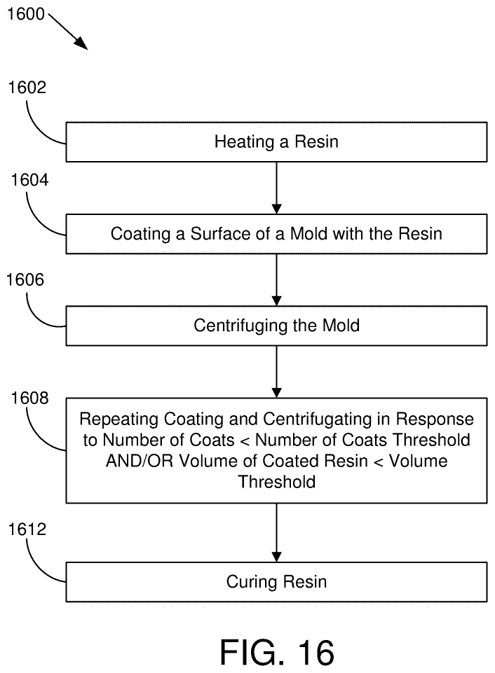

FIG. 16 is a flow chart of a method 1600 of coating a mold (e.g., a mold 1502) for forming a lens 100, according to the present disclosure. In some examples, the method 1600 is performed after forming the mold 1502 and prior to forming the lens body 102 within the mold 1502 to help reduce aberrations in the surface 1504 of the mold 1502 and, consequently, in the surface 108 of the lens body 102. Molds such as mold 1502 can be susceptible to the formation of irregularities on their surfaces 1504 during the manufacturing process. Such irregularities include, for example, layer lines, aberrations, indentations, rough portions, and/or protrusions. Irregularities in the surfaces of the mold 1502 can yield irregularities in the surfaces of the lens body 102, since the lens body 102 is formed in the mold 1502. As such, examples of the present disclosure include a method 1600 that helps to reduce such irregularities in the mold 1502.

In some examples, the method 1600 includes heating 1602 a resin. In some examples, heating 1602 the resin includes heating the resin to a temperature of no less than 60 degrees Celsius and not greater than 125 degrees Celsius. In some examples, heating 1602 the resin includes heating the resin to a temperature of not less than 90 degrees Celsius and not greater than 100 degrees Celsius. In some examples, the resin is a monomer, such as an ultraviolet (“UV”) photopolymer.

In some examples, the method 1600 includes coating 1604 a surface 1504 of the mold 1502 with the resin. In some examples, the surface 1504 is a concave surface of a larger cavity of the mold 1502. In some examples, coating 1604 the surface 1504 with the resin includes drop coating the resin onto the surface 1504. In some examples, coating 1604 the surface 1504 with the resin includes dropping multiple drops of the resin onto the surface 1504.

In some examples, the method 1600 includes centrifuging 1606 the mold 1502. In some examples, centrifuging 1606 the mold 1502 includes rotating the mold 1502 about an axis. In some examples, the axis is central to the mold 1502. Some examples include centrifuging 1606 the mold 1502 using a centrifuge and/or a rotor. In some examples, the centrifuging 1606 occurs at a centrifugation speed of not less than 1800 revolutions per minute (“rpm”) and not greater than 2200 rpm. In some examples, the motion creates a centrifugal force that distributes the resin and causes the resin to fill any indentations in the concave surface 1504.

In some examples, centrifuging 1606 the mold 1502 includes centrifuging the mold 1502 for an initial period of approximately 10 seconds, or not less than 8 seconds and not greater than 12 seconds. Some examples of the method 1600 include selecting a centrifugation period based at least in part on a centrifugation speed. In some examples, a ratio of a centrifugation period in seconds to a centrifugation speed in rpm is in a range of 1 to 150 and 1 to 250.

In some examples, the method 1600 includes repeating 1608 coating 1604 the surface 1504 of the mold 1502 with the resin and centrifuging 1606 the mold 1502. In some examples, the repeating 1608 is in response to: (i) a number of coats of the resin on the surface 1504 being below a number of coats threshold and/or (ii) a volume of the applied resin being below a volume threshold. In some examples, the volume threshold is not less than 2.5 microliters (μL) and not greater than 7.5 μL. In some examples, the volume threshold is not less than 4.5 μL and not greater than 5.5 μL. In some examples, the volume threshold is approximately 5 μL. In some examples, coating 1604 the surface 1504 with the resin includes applying a first coat of the heated resin, and the repeating 1608 includes applying a second coat of the heated resin. In some examples, the number of coats threshold is not less than 2 coats and not greater than 4 coats. In some examples, the repeating 1608 includes centrifuging the mold again after applying the second coat.

In some examples, the method 1600 includes curing 1612 the resin to the surface 1504. Some examples including curing 1612 the resin to the surface 1504 for a period of not less than 4 seconds and not greater than 6 seconds. In some examples, the period is approximately 5 seconds.

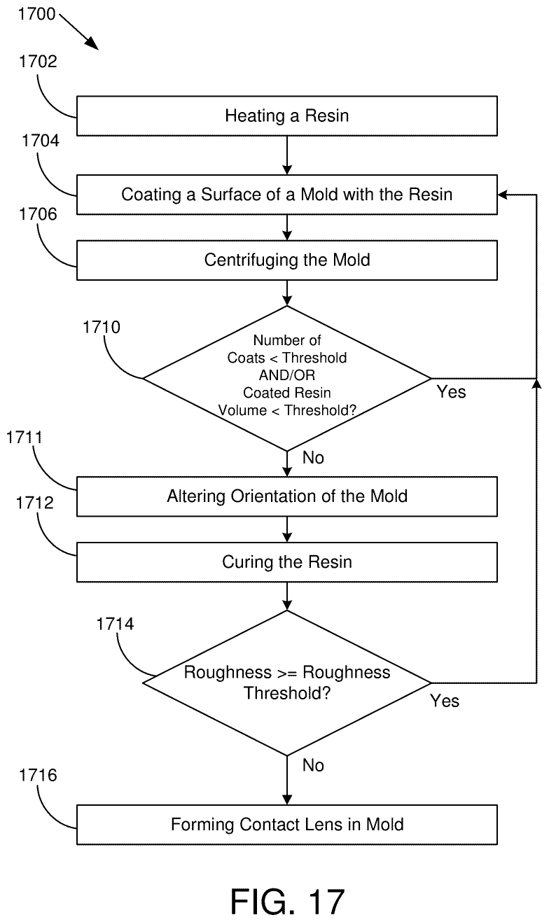

FIG. 17 illustrates a flow chart of an additional method 1700 for coating a mold 1502 for forming a contact lens body 102. In some examples, the method 1700 includes steps of the method 1600, such as heating 1702 a resin 1802, coating 1704 a surface 1504 of the mold 1502 with the resin 1802, centrifuging 1706 the mold 1502, repeating the coating 1704 and/or centrifuging 1706 in response to determining 1710 that the number of coats the resin is below a number of coats threshold and/or that the coated resin volume is below a volume threshold, and curing 1612 the resin 1802.

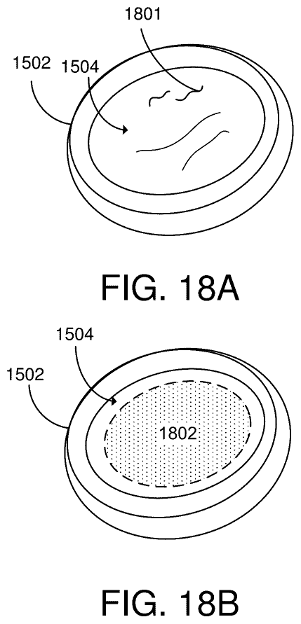

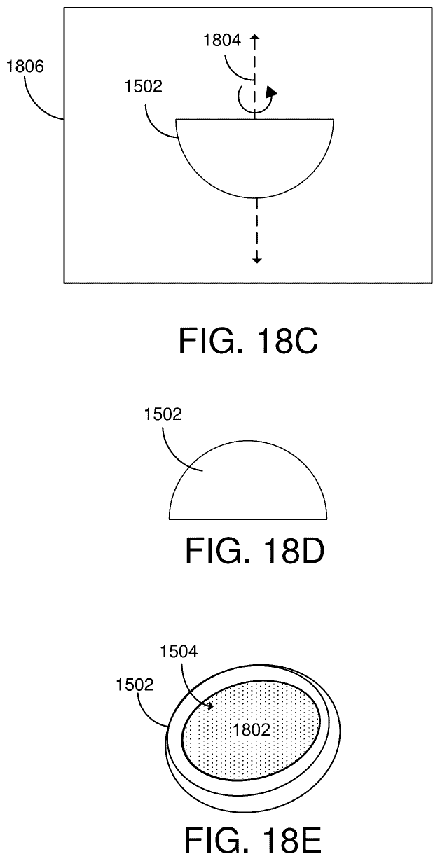

In some examples, the method 1700 includes altering 1711 an orientation of the mold 1502 between the centrifuging 1706 and the curing 1712. As shown in FIG. 18C, in some examples, the method 1700 includes centrifuging 1706 with the concave surface 1504 facing upwards and curing 1712 the resin 1802 to the surface 1504 with the surface 1504 facing downwards. In some examples, changing 1711 the orientation of mold 1502 includes changing the orientation of the mold, about an axis substantially perpendicular to the axis 1804 of centrifugation, by not less than 120 and not greater than 240 degrees. In some examples, changing 1711 the orientation of the mold 1502 includes changing 1711 the orientation by approximately 180 degrees.

In some examples, the method 1700 includes determining 1714 a roughness of the surface 1504 after curing the resin 1802 to the surface 1504. In some examples, the determining 1714 includes comparing the roughness to a roughness threshold. In some examples, the comparison is done through optical analysis. In some examples, the comparison is done by comparing images of the surface 1504 to a model. If the roughness is greater than or equal to the threshold, the method 1700 includes repeating the steps of coating 1704 the surface, centrifuging 1706, altering 1711 the orientation of the mold 1502, and/or curing 1712 the resin 1802.

In some examples, the method 1600 includes forming a contact lens body 102, as illustrated in FIGS. 15A-D, by depositing material onto the concave surface 1504 of the lens mold 1502 after curing the resin used to form the mold. In some examples, forming the contact lens body 102 includes curing the material deposited on the concave surface 1504. In some examples, depositing the material onto the surface 1504 to form the lens body 102 is in response to determining 1714 that the roughness of the surface 1504 is below the roughness threshold.

FIGS. 18A-D are schematic diagrams of a method 1800 for coating a surface 1504 of a mold 1502 for forming a contact lens body 102. FIGS. 18A-D illustrate examples of the methods 1600 and 1700. As shown in FIG. 18A, a mold 1502 for forming a contact lens body 102 includes a surface, such as the concave surface 1504. As shown in FIG. 18A, in some examples, the concave surface 1504 initially includes a number of irregularities 1801, such as ridges.

In some examples, the mold 1502 includes two parts coupled to each other in a clamshell configuration. In some examples, a first part includes the concave surface 1504. In some examples, the first part is a lower part of the mold 1502. In some examples, the mold 1502 includes a second part with a concave surface that faces the concave surface 1504.

As shown in FIG. 18B, the method 1800 includes coating the surface 1504 with a resin 1802. As shown in FIG. 18B, in some examples, the resin 1802 does not completely cover the surface 1504 and/or is not distributed evenly throughout the surface 1504 initially.

As shown in FIG. 18C, in some examples, the method 1800 includes centrifuging the mold 1502 by rotating the mold 1502 about an axis 1804. In some examples, the method 1800 includes centrifuging the mold 1502 via a centrifuge 1806, which houses the mold 1502 during the centrifugation process and includes a rotor configured to cause the mold 1502 to rotate about the axis 1804. In some examples, the method 1800 includes rotating the mold 1502 around an axis of the centrifuge 1806 rather than around an axis 1804 of the mold 1502. In some examples, the centrifuge includes a holder configured to receive the mold 1502. In some examples, the holder is positioned on an arm configured to spin the mold 1502 around the axis of the centrifuge 1806 to help deposit the resin into indentations in the mold 1502 and distribute the resin across the surface 1504.

As shown in FIG. 18D, in some examples, the method 1800 includes altering an orientation of the mold 1502 and curing the resin 1802 within the mold 1502. In some examples, the concave surface 1504 of the mold 1502 faces upwards while the centrifugation takes place, as shown in FIG. 18C. In some examples, the concave surface 1504 faces downwards while the resin 1802 is being cured to the concave surface 1504.

As shown in FIG. 18E, in some examples, the centrifugation and curing more evenly distributes the cured resin 1802 throughout the concave surface 1504, helping to reduce the risk of exposed irregularities 1801. In some examples, the concave surface 1504, after performance of the method 1800, is less prone to irregularities 1801.

The present invention may be embodied in other specific forms without departing from its spirit or essential characteristics. The described examples are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A lens comprising:
a body of a contact lens, the body comprising a side; and
a plurality of nodes formed onto the side, each node of the plurality of nodes having a parameter value that is based at least in part on a reduction of transmission of light through the lens at one or more wavelengths, the one or more wavelengths selected to alter a color perception and/or color discriminability of a person, the parameter value comprising a value of: a distance between the node and an adjacent node of the plurality of nodes, a shape of the node, a dimension of the node, a configuration of an arrangement in which the plurality of nodes are arranged on the side, a composition of a material coating the node, and/or a material composition of the node.

2. The lens of claim 1, wherein the one or more wavelengths comprise a wavelength range that is based at least in part on a color vision deficiency (“CVD”) profile of the person.

3. The lens of claim 2, wherein the plurality of nodes are separated from each other at a distance that is based at least in part on a wavelength within the wavelength range.

4. The lens of claim 2, wherein the CVD profile comprises a deficiency of the person in perceiving light within a tested wavelength range of light and a deficiency of the person in discriminating between light of different wavelengths within the tested wavelength range of light.

5. The lens of claim 4, wherein the wavelength range is based at least in part on the deficiency of the person in perceiving light within the tested wavelength range of light and the deficiency of the person in discriminating between light of different wavelengths within the tested wavelength range of light.

6. The lens of claim 1, each of the plurality of nodes comprising an ellipse shape, the ellipse shape comprising an aspect ratio, wherein the dimension comprises the aspect ratio.

7. The lens of claim 1, wherein the plurality of nodes are separated from each other a distance not less than 10 nanometers (“nm”) and not greater than 1000 nm away from each other.

8. The lens of claim 1, wherein the body includes a plurality of cavities and each node of the plurality of nodes is located within a cavity of the plurality of cavities.

9. The lens of claim 1, at least one node of the plurality of nodes comprising an ellipse shape, wherein a length of a minor axis of the ellipse shape is not less than 5 nm and not greater than 150 nm.

10. The lens of claim 1, wherein the arrangement of the plurality of nodes comprises a substantially square array on the side and the side comprises a convex side.

11. The lens of claim 1, wherein each node of the plurality of nodes comprises a metallic material.

12. An apparatus comprising:
a perception module configured to determine a person's perception of light at one or more wavelengths across a range of tested wavelengths;
a discriminability module configured to determine a person's discriminability between light across the range of tested wavelengths;
a transmission reduction module configured to determine a wavelength range selected to alter a color perception and/or a color discriminability of the person based at least in part on the person's perception of light, the person's discriminability between light across the range of tested wavelengths, and the one or more wavelengths; and
a node module configured to determine, for each node of a plurality of nodes and based at least in part on the wavelength range, a parameter value, the parameter value comprising a value of: a distance between the node and an adjacent node of the plurality of nodes, a shape of the node, a dimension of the node, a configuration of an arrangement of nodes of the plurality of nodes, a composition of a material coating a node, and/or a material composition of the node; and
a formation module configured to perform the following:
form the node based at least in part on the shape of the node, the dimension of the node, the composition of the material coating the node, and/or the material composition of the node;
form a plurality of cavities in a substrate based at least in part on the distance between the node and the adjacent node of the plurality of nodes and/or the configuration of the arrangement of nodes of the plurality of nodes; and/or
cause the plurality of nodes to be deposited onto a substrate based at least in part on the distance between the node and the adjacent node of the plurality of nodes and/or the configuration of the arrangement of nodes of the plurality of nodes,
wherein at least a portion of said modules comprise one or more of hardware circuits, programmable hardware circuits and executable code, the executable code stored on one or more computer readable storage media.

13. The apparatus of claim 12, wherein:
the transmission reduction module is further configured to determine a percentage of transmission reduction of light within the wavelength range; and
the node module is further configured to determine the value of the parameter based at least in part on the percentage.

14. The apparatus of claim 12, further comprising a simulation module configured to generate a number of simulations of transmission of light through a lens, each simulation of the number of simulations simulating nodes of a test value of one or more test values of the parameter, wherein the node module is further configured to determine the value of the parameter based at least in part on results from the number of simulations.

15. The apparatus of claim 12, wherein the node module is further configured to determine the value of the parameter based at least in part on the person's perception of light across the range of tested wavelengths and on the person's discriminability between light of different wavelengths across the range of tested wavelengths.

16. The apparatus of claim 12, wherein the substrate comprises a contact lens body, a substrate of a stamp for forming cavities in a contact lens body, a substrate of a stamp for forming a mold for forming a contact lens, and/or a mold for forming a contact lens.

17. A method comprising:
determining, using a perception module, a person's perception of light at one or more wavelengths across a range of tested wavelengths;
determining, using a discriminability module, a person's discriminability between light across the range of tested wavelengths;
determining, using a transmission reduction module, a wavelength range to alter a color perception and/or a color discriminability of the person based at least in part on the perception of light, the discriminability between light, and the one or more wavelengths;
determining, using a node module, for each node of a plurality of nodes and based at least in part on the wavelength range, a parameter value, the parameter value comprising a value of: a distance between the node and an adjacent node of the plurality of nodes, a shape of the node, a dimension of the node, a configuration of an arrangement of nodes of the plurality of nodes, a composition of a material coating a node, and/or a material composition of the node; and
performing at least one of the following:
forming the node based at least in part on the shape of the node, the dimension of the node, the composition of the material coating the node, and/or the material composition of the node;
forming a plurality of cavities in a substrate based at least in part on the distance between the node and the adjacent node of the plurality of nodes and/or the configuration of the arrangement of nodes of the plurality of nodes;
and/or causing the plurality of nodes to be deposited onto a substrate based at least in part on the distance between the node and the adjacent node of the plurality of nodes and/or the configuration of the arrangement of nodes of the plurality of nodes.

18. The method of claim 17, wherein the substrate is separate from a body of a lens, and the method further comprises transferring the plurality of nodes onto a side of a body of a lens.

19. The method of claim 17, wherein determining the person's discriminability between light across the range of tested wavelengths comprises determining a sensitivity of cones in the person's retina to incident irradiation across the range of tested wavelengths, and the range of tested wavelengths comprises a range within the visible spectrum.

20. The method of claim 17, further comprising determining the person's perception of light and the person's discriminability between light using an anomaloscope.
